(19) United States
(12) Patent Application Publication
(10) Pub. No.: US 2024/0252240 A1
(43) Pub. Date: Aug. 1, 2024

(54) Energy Profile Regulating Waveguide, and Laser-Based Medical Apparatus having such Waveguide

(71) Applicant: SQUALUS MED LTD., Harutzim (IL)

(72) Inventors: Moshe Eshkol, Harutzim (IL); Gil Shapira, Tel Aviv (IL)

(21) Appl. No.: 18/290,030

(22) PCT Filed: May 22, 2022
(86) PCT No.: PCT/IL2022/050534

§ 371 Date: Nov. 9, 2023

Related U.S. Application Data
(60) Provisional application No. 63/192,239, filed on May 24, 2021.

(51) Int. Cl. A61B 18/24; A61B 18/00; A61B 18/22

(52) U.S. Cl. CPC A61B 18/24; A61B 2018/00023; A61B 2018/00285; A61B 2018/00547; A61B 2018/2261

(57) ABSTRACT

Energy profile regulating waveguide, and laser-based medical apparatus having such waveguide. In a medical device, an optical fiber or a waveguide includes, at an internal side thereof, a refracting optical element or a deflecting optical element or both; which diverts laser energy, that propagate along the optical fiber or waveguide, to exit therefrom sideways through a side-wall and to provide laser energy to an in-vivo location that is located sideways relative to the general propagation direction of the laser energy within the optical fiber or waveguide. Optionally, some of the laser beams that propagate within the optical fiber or waveguide, exit from it sideways and intersect or super-impose at a focal point or focal region, at which a laser-based medical procedure is performed.

### CROSS-REFERENCE TO RELATED APPLICATIONS

This patent application claims priority and benefit from U.S. 63/192,239, filed on May 24, 2021, which is hereby incorporated by reference in its entirety.

### FIELD

Some embodiments are related to the field of medical devices.

### BACKGROUND

A waveguide is a structure that guides waves, such as electromagnetic waves or sound waves, with minimal loss of energy. For example, a hollow conductive metal pipe may be used to carry high frequency radio waves. Dielectric waveguides may be used at higher radio frequencies. Transparent dielectric waveguides and optical fibers may serve as waveguides for light. In acoustics, air ducts and horns may be used as waveguides for sound (e.g., in musical instruments and loudspeakers). Specially-shaped metal rods may conduct ultrasonic waves in ultrasonic machining.

### SUMMARY

Some embodiments provide an energy profile regulating waveguide, and laser-based medical devices having such waveguide. For example, in accordance with some embodiments, a medical device has an optical fiber or a waveguide; which includes, at an internal side thereof, a refracting optical element and/or a deflecting optical element; which diverts laser energy, that propagates along the optical fiber or waveguide, to exit therefrom sideways through a side-wall and to provide laser energy to an in-vivo location that is located sideways relative to the general propagation direction of the laser energy within the optical fiber or waveguide (e.g., instead of guiding the entirety of propagating laser energy to exit in a forward direction, via a tip or cap at the distal end of the optical fiber or waveguide). Optionally, some of the laser beams that propagate within the optical fiber or waveguide, exit from it sideways and intersect or super-impose at a focal point or focal region, at which a laser-based medical procedure is performed.

Some embodiments may provide other and/or additional benefits and/or advantages.

### BRIEF DESCRIPTION OF THE DRAWINGS

FIG. 1 is a schematic illustration of a portion of a waveguide, particularly for propagating light or laser-based energy for medical treatment purposes, in accordance with some demonstrative embodiments.

FIG. 2 is a schematic illustration of another portion of a waveguide, in accordance with some demonstrative embodiments.

FIG. 3 is a schematic illustration of a portion of a waveguide, particularly for propagating light or laser-based energy for medical treatment purposes, in accordance with some demonstrative embodiments.

FIG. 4 is a schematic illustration of a portion of a waveguide, demonstrating schematically an optical view in accordance with some demonstrative embodiments.

FIG. 5 is a schematic illustration of a portion of a waveguide, demonstrating schematically areas having different light-based energy profiles, in accordance with some demonstrative embodiments.

FIG. 6 is a schematic illustration of a portion of a waveguide, in accordance with some demonstrative embodiments.

FIG. 7 is a schematic illustration of a portion of a waveguide, in accordance with some demonstrative embodiments.

FIG. 8 is a schematic illustration of a portion of a waveguide, in accordance with some demonstrative embodiments.

FIGS. 9A and 9B are schematic illustrations of a portion of a waveguide, demonstrating modifications to the energy profile due to controlled changes in a controlled liquid or gas or fluid environment, in accordance with some demonstrative embodiments.

FIG. 10 is an illustration of an isometric view of a portion of a waveguide with directional emission of energy, in accordance with some demonstrative embodiments.

FIGS. 11-15 are schematic illustrations of cross-sectional views of some waveguides, in accordance with some demonstrative embodiments.

FIGS. 16-17 are schematic illustrations of cross-sectional views of additional waveguides, demonstrating the integration of two or more of features in a single device, in accordance with some demonstrative embodiments.

FIGS. 18A and 18B are schematic illustrations of a portion of a waveguide or optical fiber, enclosed in (or surrounded by) an inflatable balloon, in accordance with some demonstrative embodiments.

FIG. 19A is a schematic illustration of a medical device, in accordance with some demonstrative embodiments.

FIG. 19B is a schematic illustration of a medical device, in accordance with some demonstrative embodiments.

FIG. 19C is a schematic illustration of a medical device, in accordance with some demonstrative embodiments.

FIG. 19D is a schematic illustration of a medical device, in accordance with some demonstrative embodiments.

FIG. 20A is a schematic illustration of a medical device, in accordance with some demonstrative embodiments.

FIG. 20B is a schematic illustration of a medical device, in accordance with some demonstrative embodiments.

FIG. 20C is a schematic illustration of a medical device, in accordance with some demonstrative embodiments.

### DETAILED DESCRIPTION OF SOME DEMONSTRATIVE EMBODIMENTS

It is clarified that the drawings are not necessarily drawn to scale. Some components and elements in the drawings are intentionally or exaggeratedly smaller or larger or shorter or longer than they are in real life, in order to more clearly show and demonstrate some structural and/or functional features of some embodiments.

Some embodiments provide an energy profile regulating waveguide, or a waveguide capable of regulating or modifying its energy profile or its energy output characteristics in accordance with a particular or pre-defined energy profile modification scheme or in accordance with a particular pre-defined configuration. Some embodiments include laser-based devices, and particularly medical laser-based devices, which include or incorporate or utilize such waveguide. Some embodiments enable fiber optic energy output profile modifications, which may be utilized for treatment with light or with light energy of some medical conditions, such as various intra-corporeal disease states.

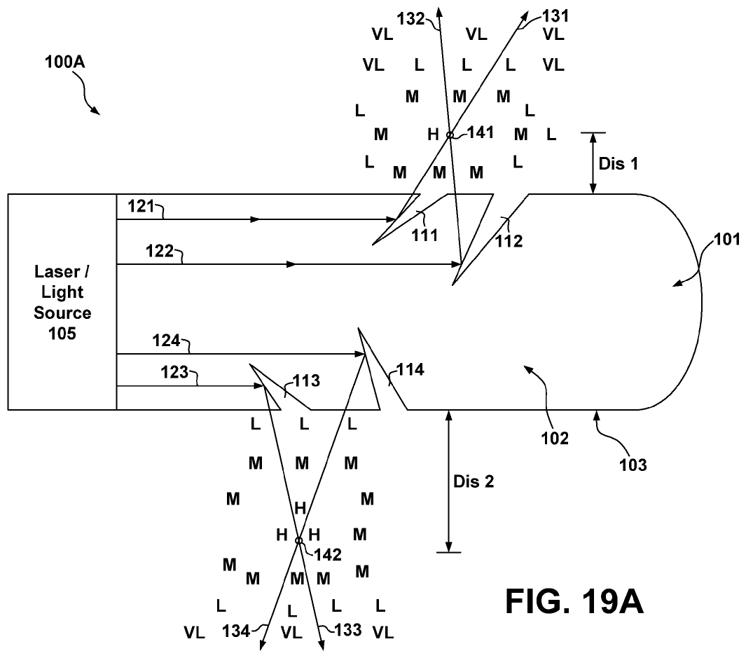

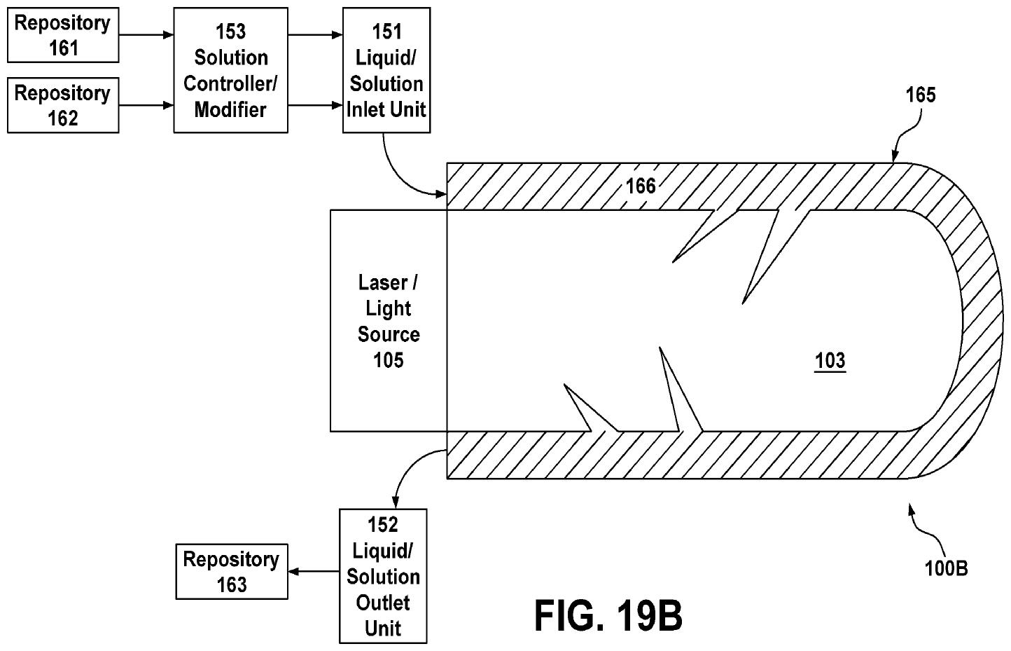

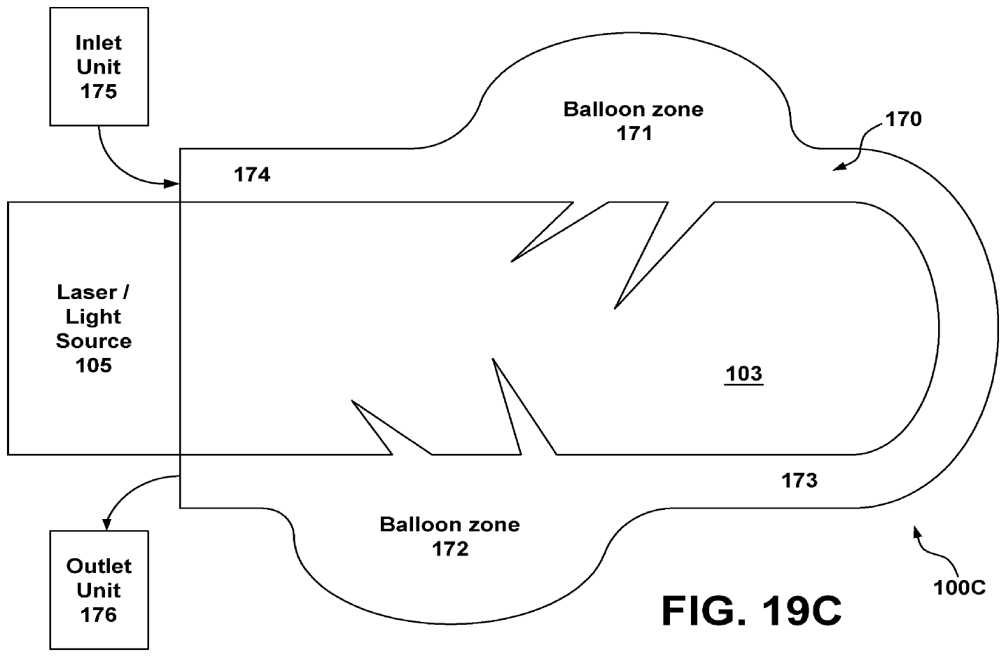

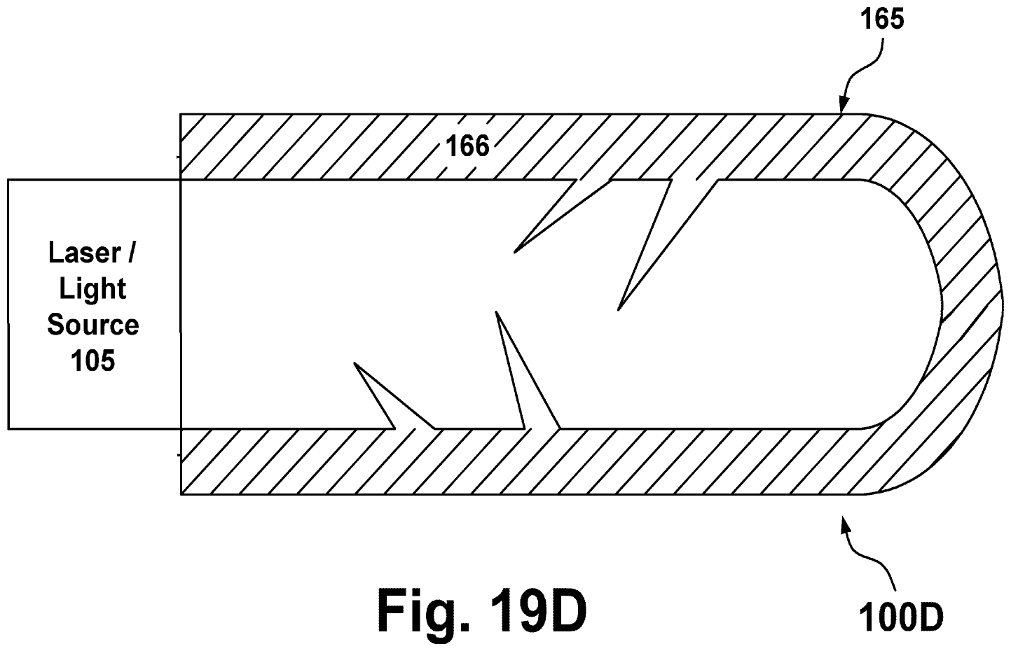

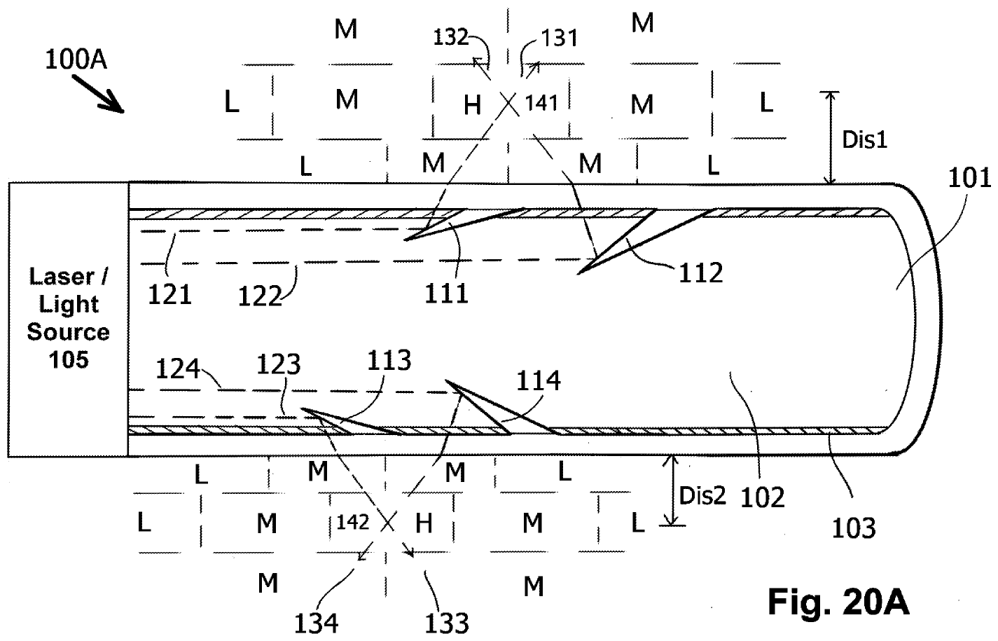

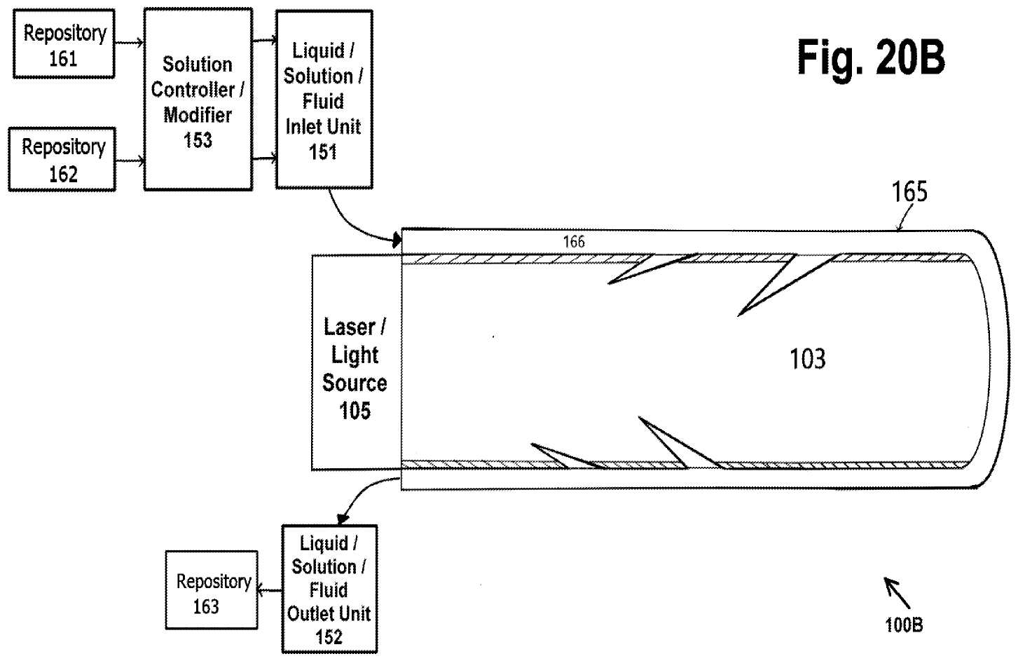

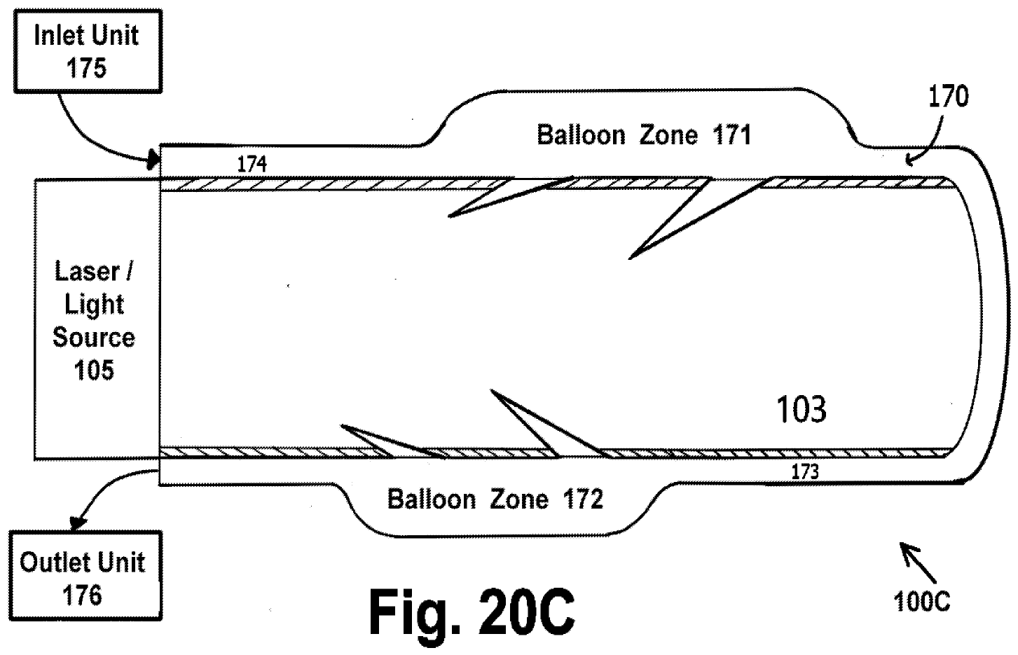

Reference is made to FIG. 19A and similarly to FIG. 20A, which are schematic illustrations of a medical device 100A, in accordance with some demonstrative embodiments. Device 100A may be, for example, a catheter configured to perform light-based treatment or laser-based treatment of a particular in-vivo region-of-interest. For example, device 100A may be inserted into a patient's body, typically when the patient is sedated or under anesthesia, or during surgery or other medical procedure (e.g., endoscopy, anoscopy, arthroscopy, bronchoscopy, colonoscopy, colposcopy, cystoscopy, esophagoscopy, gastroscopy, laparoscopy, laryngoscopy, neuro-endoscopy, proctoscopy, sigmoidoscopy, thoracoscopy); and may be guided, typically in a flexible or semi-flexible manner, towards the region-of-interest (e.g., a tumor or a lesion), or may be otherwise brought to the region-of-interest (e.g., directly, if there is physical access to that region-of-interest). Device 100A may then generate and output energy, particularly light-based energy or optical energy or laser-based energy. In accordance with some embodiments, the energy level and direction are controllable and/or modifiable, in order to ensure that the energy treats particularly (or only, or exclusively, or selectively) the region-of-interest, and/or to ensure that the energy does not treat (at all; or, treats minimally, or affects minimally) other region(s) which are not within the region-of-interest (e.g., are adjacent to it, or are neighboring it or surrounding it, or are in proximity to it).

Device 100A includes a laser/light source 105, which generates laser or light beams or rays. For demonstrative purposes, four such rays 121-124 are shown. The laser or light rays enter a waveguide 103 or an optical fiber or a fiber optic, typically implemented as a thin, elongated, generally hollow, flexible tube or pipe, having a length that is typically between 30 to 500 centimeters, or between 10 to 500 centimeters (e.g., for treating prostate conditions, the device may be approximately 20 or 200 centimeters long), or between 10 to 1,000 centimeters, or between 10 to 2,000 centimeters (e.g., for performing certain medical conditions, particularly with Magnetic Resonance Imaging (MRI), a 10-meter or 15-meter or even 20-meter device may be used); and is typically cylindrical or generally cylindrical, and has a circular or generally circular cross-section, typically having a diameter in the range of 0.5 to 10 millimeters, or in the range of 0.2 to 10 millimeters, or in the range of 0.1 to 10 millimeters.

For demonstrative purposes, item 103 is referred to herein as “waveguide”, although it may also be referred to as “optical fiber” or “fiber optic”. In some embodiments, waveguide 103 is enclosed within a capsule or a jacket or other envelope or sleeve or protective layer; and typically has a central core that is surrounded by a cladding layer or a clad; these are not shown in FIG. 19A, in order to not over-crowd the drawing.

In some embodiments, waveguide 103 (or the catheter in which it is enclosed) ends with a tip/cap zone 101, which may be dome-shaped or may be tapered (e.g., to penetrate through body tissue). The tip or cap zone is small relative to the entire length of the waveguide; for example, the tip/cap zone may occupy less than 1% of the entire length of the waveguide, and a non-cap/non-tip zone 102 may occupy at least 99% of the length of the waveguide. In some embodiments, particularly when the entire length of the medical device is relatively short (e.g., less than 30 centimeters, or less than 20 centimeters, or less than 15 centimeters), the tip/cap zone 101 may occupy less than 5% of the entire length of the waveguide, or may occupy less than 3% of the entire length of the waveguide, or may occupy less than 2% of the entire length of the waveguide, or may occupy approximately 0.5 to 5 percent of the entire length of the waveguide; and the remainder is the non-cap/non-tip zone 102. The tip/cap zone 101 is different from the non-tip/non/cap zone of the waveguide, by at least one property; for example, by shape, having a tapered or semi-spherical shape at the tip/cap zone, in contrast with the generally cylindrical shape of the non-tip/non-cap zone of the waveguide; and/or by width or thickness, for example, such that the tip/cap zone (or at least a portion or a region thereof) is thinner or is less thick or is less wide relative to the non-tip/non-cap zone of the waveguide; or the like.

In accordance with some embodiments, a first side or region or area of waveguide 103 includes a first set of optical elements; and a second, different, side or region or area of waveguide 103 includes a second set of optical elements, which may be generally similar or may be identical to those of the first side, or may be different from those of the first side. For demonstrative purposes, some portions of the discussion and/or some of the drawings may show or may discuss two such regions or segments or the same waveguide; however, some embodiments may utilize only one single region or segment in a waveguide, or may utilize three or more such regions or segments in a waveguide; and similarly, some embodiments may have such segments or regions only on one side thereof, or may have them at two opposing sides thereof, or at two non-opposing sides thereof, or at three (or more) sides thereof.

For demonstrative purposes, a first side of waveguide 103 is its top side in this drawing, and it includes two optical elements 111-112; and, a second side of waveguide 103 is its bottom side in this drawing, and it includes two other optical elements 113-114. For example, each one of optical elements 111-114 is an inwardly-facing or inwardly-protruding or inwardly-directed wedge or optical hurdle or tooth or optical redirector element (e.g., mirroring element, mirror, micro-mirror, flat mirror, planar mirror, non-planar mirror, non-planar reflecting element, curved mirror, convex mirror, concave mirror, diffuser or optical diffuser or light diffuser, reflective element, refractive element, prism, lens, micro-lens, or the like) able to deflect and/or refract an incoming ray or beam (or a portion thereof) towards a particular direction (or towards multiple directions). Optionally, each of optical elements 111-114 may have particular structural and/or optical properties or functionalities, which may be configured by forming such element from a particular material, and/or by coating such elements (or portions thereof) with a particular coating, and/or by setting or modifying or configuring or adjusting the index of refraction of an external coating or sleeve or medium or encapsulating element, and/or by structuring or forming such element to have a particular three-dimensional shape and/or volume and/or size and/or contour and/or slanting structure, and/or by setting the location of one or more of such elements and/or the distance among them and/or the number of such elements.

For example, due to the position, location, size, slanting, structure, distancing, forming material, and/or coating of each optical element 111-114 (or of an external medium or sleeve or encapsulating element), one or more of the incoming rays 121-124 may be refracted or deflected; particularly, away from the long axis of waveguide 103, or away from the longitudinal dimension or axis of waveguide 103; or towards a point or a region that is located sideways relative to the longitudinal dimension of waveguide 103, and not towards a point or a region that are located in front of (or ahead of) the tip/cap zone 101 of waveguide 103.

For example, incoming ray (or in-waveguide ray) 121 is deflected or refracted by the surface of optical element 111, and/or due to one or more properties of that optical element 111 (e.g., its size, length, slanting, location, coating, or the like), and exits the waveguide 103 (and the medical device 100A) sideways as outgoing ray 131. Additionally, incoming ray (or in-waveguide ray) 122 is deflected or refracted by the surface of optical element 112, and/or due to one or more properties of that optical element 112 (e.g., its size, length, slanting, location, coating, or the like), and exits the waveguide 103 (and the medical device 100A) sideways as outgoing ray 132.

The outgoing rays 131 and 132 intersect or meet or collide or super-impose at focal point 141 (which may be a singular focal point, particularly when demonstrating the intersection of two rays; or, in some embodiments, may be a focal area or a focal zone or a focal region, typically shaped as a rectangle or square or diamond or quadrilateral or other polygon or as a circle or an oval, demonstrating the area in which multiple rays intersect); which is located at a distance Dis1 from waveguide 103 (or, from the external side of the envelope or capsule or catheter in which waveguide 103 is encapsulated). Accordingly, focal point 141, or similarly a focal region or focal area, and its immediately adjacent area or region, are High energy areas or regions, and are denoted with H for High in the drawing. Areas or regions that lead from the external envelope of waveguide 103 to focal point 141, in which the outgoing rays 131-132 do not yet intersect, are Low energy regions or are Medium energy regions. After meeting or intersecting or super-imposing at the High energy focal point 141, the outgoing rays 131-132 diverge; and they travel through additional Medium energy regions, and then through Low energy zones and/or Very Low energy zones.

Similarly, incoming ray (or in-waveguide ray) 123 is deflected or refracted by the surface of optical element 113, and exits the waveguide 103 (and the medical device 100A) sideways as outgoing ray 133. Additionally, incoming ray (or in-waveguide ray) 124 is deflected or refracted by the surface of optical element 114, and exits the waveguide 103 (and the medical device 100A) sideways as outgoing ray 134. The outgoing rays 133 and 134 intersect or meet or super-impose at focal point 142, or at a focal area or focal region nearby; which is located at a distance Dis2 from waveguide 103 (or, from the external side of the envelope or capsule or catheter in which waveguide 103 is encapsulated). Accordingly, focal point 142, or a nearby focal region or focal area, and its immediately adjacent area or region, are High energy areas or regions, and are denoted with H for High in the drawing. Areas or regions that lead from the external envelope of waveguide 103 to focal point 142, in which the outgoing rays 133-134 do not yet intersect, are Low energy regions or are Medium energy regions.

After meeting or intersecting or colliding or or super-imposing at the High energy focal point 142, the outgoing rays 133-134 diverge; and they travel through additional Medium energy regions, and then through Low energy zones and/or Very Low energy zones. As mentioned, each one of the High energy focal points (141, 142) is not necessarily a singular point; but rather, it may be a High energy focal zone or a High energy focal area or region, through which numerous such rays intersect at numerous intersection points or super-position points that are neighboring each other or that are in the immediate vicinity of each other.

It is noted that such High energy focal point(s) or focal area(s) or focal zone(s) or region(s), may be located at other suitable locations or spatial locations relative to the waveguide; for example, they may be located near and externally to the tip zone or the cap zone, or they may be located externally to or near several different areas of the waveguide. For example, in some embodiments, K1 such focal points or focal zones or focal areas may be located at a first side of the waveguide; and K2 such focal points or focal zones or focal areas may be located at a second, different, side of the waveguide (e.g., a generally opposing side; or a non-opposing side); and K3 such focal points or focal zones or focal areas may be near and externally to the tip zone or the cap zone of the waveguide; wherein K1 and K2 and K3 are pre-defined values, each one of them may be 0 or 1 or 2 or 3 or may be an integer that is typically smaller than 10 or typically smaller than 100; each one of them may be different from the other, or two (or more) of them may have the same value.

It is noted that the two distances, Dis1 and Dis2, may be identical to each other; or, in some embodiments, they may be different from each other, in order to provide two different distances (perpendicularly to the external envelope of waveguide 103) at which a High energy profile occurs. In some embodiments, optionally, waveguide 103 may comprise only one side of optical elements, rather than two sides; such as, only optical elements 111-112; such that only a single side of waveguide 103 would emit energy or rays or beam that have a single focal point of High energy at that side only.

The distances, Dis1 and/or Dis2, may be configured or determined based on the properties of optical elements 111-112 and 113-114, respectively; such as, the type of each optical element (e.g., wedge, inwardly-facing tooth, inwardly-facing protrusion, prism, lens, planar mirror, curved mirror), the slanting or the curvature of each optical element, the spatial shape of each optical element, the depth that each optical element penetrates or blocks into the core of waveguide 103, the amount of rays or beams that each optical element is able to re-direct, the surface area of the optical element, the surface area of the particular surface of the optical element that is facing towards the direction from which rays or beams are incoming, the material(s) from which that optical element is formed, the material(s) that form one or more sleeves or encapsulation layer(s) or medium(s) that surround the waveguide or the optical fiber and/or the encapsulate the waveguide or the optical fiber (e.g., and which may have a particular index of refraction, which may optionally be set or configured in order to achieve a particular optical characteristic), the coating of each optical element, the number of optical elements in each segment or region of waveguide (e.g., two optical elements in series on the same side; or three optical elements in series on the same side; or N optical elements in series on the same side), the longitudinal distance interval that separates between two consecutive optical elements on the same side (e.g., the distance between optical element 111 and optical element 112), and/or other structural parameters which may be configured or set in advance, and/or the wavelength of the incoming light rays or laser rays (e.g., which may impact the location or the distance of each focal point or focal area, due to change in the absorption of energy by the tissue of the relevant body organ).

Reference is made to FIG. 19B and FIG. 20B, which are schematic illustrations of a medical device 100B, in accordance with some demonstrative embodiments. Device 100B may be generally similar to device 100A of FIG. 19A; however, device 100B further includes a Solution Sleeve 165 which surrounds or encapsulates waveguide 103 (or at least a portion thereof). Solution Sleeve 165 may be a thin, elongated, container or channel or repository able to store therein a solution 166 or other material (e.g., liquid, gas, fluid). For example, two or more repositories 161-162 may store two or more materials (e.g., water in repository 161; sugar in repository 162); and a Solution Controller/Modifier Unit 153 operates to obtain (e.g., pump out, suck out) those two materials from the repositories 161-162 in accordance with a particular ratio (e.g., water to sugar ratio of 100 to 3), to mix them, and to provide the mixed solution via a Liquid Solution Inlet Unit 151 into the Solution Sleeve 165.

The solution 166 causes a modification to the deflection or refraction of the outgoing rays (rays 131-134 shown in FIG. 19A), since it is a different medium through which those rays travel or pass; and the solution further causes a modification to the distance (Dis1 or Dis2) of the High energy focal point (141 and/or 142) from waveguide 103.

Optionally, the solution 166 or portions thereof may be selectively and controllably removed from the Solution Sleeve 165, using a controlled component such as a Liquid/Solution Outlet Unit 152 (e.g., using a valve or pump or suction unit), and may be disposed or discarded into a collection repository 163; and optionally, a new solution or a replacement solution or a modified solution may be prepared by the Solution Controller/Modifier Unit 153 and may be injected or introduced into the Solution Sleeve 165; thereby enabling the user of device 100B to modify the distance or location or the High energy focal point (141 and/or 142).

Such controlled and user-selected modification may be performed prior to insertion of the medical device 100B into a patient's body; and/or it may be performed in real time or in near real time, during surgery, or while the medical treatment is ongoing, or while at least a portion of device 100B is in vivo, or while waveguide 103 or a portion thereof is in vivo, to thereby enable real time or near real time modification or adjustment of the spatial location or the distance of the High energy focal point or focal zone or focal area, and to enable movement or relative movement of such High energy focal point or focal zone or focal area in order to gradually cover or reach a plurality of regions-of-interest or areas-of-interest for medical treatment purposes.

In some embodiments, optionally, repositories 161 and 162, as well as 163, may be implemented as a closed-loop; in some embodiments, two or more of the repositories may be implemented as a single common repository associated with a closed-loop mechanism that enables transport of fluid from the repository and then back into the same repository. Optionally, a heat exchange unit may be used, and/or a cooling unit may be used; for example, to ensure that the fluid that is transported is at a particular temperature or is within a particular range of temperatures.

Reference is made to FIG. 19C and FIG. 20C, which are schematic illustrations of a medical device 100C, in accordance with some demonstrative embodiments. Device 100C may be generally similar to device 100A of FIG. 19A; however, device 100C further includes a Balloon 170 which surrounds or encapsulates waveguide 103 or at least one or more particular regions of waveguide 103. In a demonstrative example, Balloon 170 may include one or more inflatable zones, and one or more non-inflatable zones.

For example, zone 171 (located near optical elements 111-112) is inflatable, formed of an elastic material that can be inflated and can expand its size or volume in response to injection or pumping of air or gas; and similarly, zone 172 (located near optical elements 113-114) is inflatable, formed of an elastic material that can be inflated and can expand its size or volume in response to injection or pumping of air or gas. In contrast, other zones or regions of balloon 170, such as zones 173 and 174, are non-inflatable; they may be formed of a non-elastic material, that does not inflate and does not expand its size or volume.

In some embodiments, the inflatable state of balloon zone 171, need not be identical in size or in shape to the inflatable state of balloon zone 172; for example, balloon zone 171 may inflate to P1 times its original thickness; whereas balloon zone 172 may inflate to P2 times its original thickness; wherein P1 and P2 have different values. In some embodiments, balloon zones 171 and 172 need not be symmetrical to each other; and these zones may be located at different regions along the longitudinal dimension of waveguide 103.

An Inlet Unit 175 enables an operator of device 100C to insert or inject or introduce or pump air or gas into balloon 170, in order to inflate it; in a controlled or selective manner, enabling the operator to control the level of inflation and thus also the thickness of the inflated balloon zone(s). Similarly, an Outlet Unit 176 enables an operator of device 100C to remove or pump-out or suck-out air or gas from balloon 170, in order to deflate it partially or entirely; in a controlled or selective manner, enabling the operator to control the level of deflation and thus also the remaining thickness of the previously-inflated balloon zone(s).

The inflation and deflation operations may in turn modify the size of the medium through which the outgoing rays (131-134) travel until they intersect or meet; and thus modifies, increases or decreases the distance (Dis1 or Dis2) of the relevant High energy focal point (141 or 142) from waveguide 103. Such controlled and user-selected modification may be performed in real time or in near real time, during surgery, or while the medical treatment is ongoing, or while at least a portion of device 100C is in vivo, or while waveguide 103 or a portion thereof is in vivo.

Some embodiments may combine features and/or functionalities from two or more drawings or implementations that are shown or discussed above or herein. For example, an embodiment of a medical device may comprise all three features shown in FIGS. 19A and 19B and 19C, namely, the optical elements 111-114, as well as the Solution Sleeve 165, as well as the Balloon 170. Similarly, features from other embodiments or drawings that are discussed or shown herein, may be combined together into a single implementation or a single medical device; for example, the features discussed herein which utilize curved optical elements or other types of optical elements.

It is noted that FIG. 19A, FIG. 19B and FIG. 19C show some but not necessarily all the components of the device(s) that are demonstrated in them, in order to not over-crowd the drawings and in order to keep them clear. For example, such device(s) may include a power source, a cooling unit to provide cooling to one or more of the device units, a sleeve or capsule or other encapsulating layer(s), and/or other suitable components.

Reference is made to FIG. 19D, which is a schematic illustration of a medical device 100D, in accordance with some demonstrative embodiments. Device 100D may be generally similar to device 100B of FIG. 19B; yet device 100D demonstrates that in some embodiments, the internal optical elements that are located internally within the optical fiber, may be structured and/or arranged and/or located such that they would block the entirety of the laser beams or laser energy that enters the proximal end of the optical fiber, from exiting through the distal end of the optical fiber or through its tip or cap; and/or such that all the laser beams that enter the optical fiber, are intercepted and/or diverted and/or refracted and/or diffracted and/or otherwise guided or modified during their propagation within the optical fiber or waveguide, such that all of those laser beams and all the laser energy or light energy exits sideways from a side-panel or a side-wall of the optical fiber, and such that none of the laser beams or laser energy or light energy exits the optical fiber in a forward direction or forwardly relative to the general direction of propagation; and/or such that those optical elements would cause diversion of an entirety of the laser energy or light energy, that entered into the optical fiber, such that all that energy would exit through (or would be emitted sideways from) one or more side-panels or the optical fiber, or would be emitted generally perpendicularly to (or, slanted or angularly relative to) a side-panel of the optical fiber, instead of being emitted from the tip or cap at the distal end of the optical fiber. FIG. 19D demonstrates such arrangement of optical elements, shown in conjunction with a sleeve or chamber that may contain a solution or a gas or liquid or a fluid; however, such features of FIG. 19D may similarly be combined with other features described above and/or herein, and/or with other features shown in any other drawing(s), such as (for example) with the balloon chamber or the inflatable sleeve-segment, or the like.

In accordance with some embodiments, a medical laser-based procedure or treatment may utilize laser energy generated by a laser source. For example, a probe waveguide or a fiber is positioned at a location near the portion of the body tissue that is intended to be treated with laser energy. Once the waveguide or fiber is positioned appropriately, the laser energy is delivered via the waveguide or fiber to the position to be treated or to the area-of-interest or the region-of-interest which is the subject for the intended treatment. In some medical procedures, the laser energy should be directed laterally from the tip towards the region-of-interest.

The applicants have realized that some “side fire” laser-based treatments, which utilize radially emitting laser catheters using fiber-optics, may also be used in discectomy, laparoscopy, arthroscopy, benign prostate hyperplasia, angioplasty, and related or other surgical procedures.

The “side fire” or radial laser-based fiber catheter may include a fiber optic element disposed within a cap. The fiber-optic element has a tip cut, and is highly polished at a slant angle or the shape of a cone followed by an air gap or other media or medium having a particular refraction index. When the laser energy is “fired” or emitted, the laser output beam is reflected by total internal reflection due to the difference in refractive indices of (i) the fiber core, and (ii) clad along the fiber all the way to the tip. At the tip, the polished surface or the cone or other optical element or optical structure causes the laser ray(s) to leave or exit the main axis of propagation and reflect typically by total internal reflection or lensing at an angle to the main axis of propagation. In some implementations, the tip includes a cap for protection; and the cap itself may serve for optical purposes.

The Applicants have realized that the laser energy emitted from the fiber follows the rules of point source propagation, which yield ultimately an exponential decay of the energy as one moves away from the energy source. The Applicants have further realized that the thermal profile which follows the energy, as well as absorption profiles, generally display an exponential decay profile in the radial direction from the energy source. The inherent exponential decay compels the temperature at the source to be higher than the temperature at any point at a radial distance from the source. The strength or the intensity of the decay depends mainly on the absorption coefficient of the material in which (or through which) the light propagates.

The Applicants have realized that in surgical procedures (e.g., in some interstitial procedures) or medical treatments, it is imperative not to evaporate or char or burn with laser energy nearby tissue or surrounding tissue, or body regions that are near or adjacent to the region-of-interest that is intended for treatment but are not actually within the region-of-interest. For example, such non-desired evaporation may cause increase in interstitial pressure, which in turn may cause medical complications or adverse results. Similarly, non-desired charring may cause change in the absorption coefficient, that in turn may start a cascading effect that can cause damage to the patient and also to the medical device itself. Furthermore, realized the Applicants, the limitations or constraints that pertain to the temperature at the source of the energy lead to limitations or constraints in the effective distance between the tip or edge of the laser-based device and the region-of-interest that is intended for treatment, due to the exponential decay of the energy.

Some embodiments provide an apparatus or a medical device that redistributes the energy, particularly light energy or laser-based energy, in a way that allows better adjustment of the energy (and its intensity level) to the needs in the specific treatment, and/or to the size or area or volume of the region-of-interested that is intended to be treated and/or that is expected be impacted; and further enables modification and adjustability of directionality and/or penetration depth of the laser-based energy.

For example, the apparatus or the medical device comprises optical units, with two or more optical elements in each optical unit; the number of optical units may be configured or set to achieve particular implementation goals or to facilitate a particular type of medical treatment. Each optical element redirects a portion of the laser beam or the light beam, outside (or away from) the propagation axis in a lateral angle. A multiplicity of the beams then coincide or meet at a distance from the fiber center, causing an energy focal there; and then continue to propagate at the original direction, and diverge. The distance of the focal from the fiber center can be adjusted or modified or configured, and the portion of energy from each optical element can be adjusted or modified or configured. These redistribution and adjustments enable leveling of the laser-based energy and temperature distribution in the radial direction.

In some embodiments, more than one laser source is used, with more than one wavelength. Each wavelength is characterized with a different absorption coefficient for the specific media or medium of propagation. The different absorption may be used, for example, for tailoring a different energy emission profile; since the focal points may differ for each wavelength, depending on the specific penetration depth. This structure and method allow versatile control over the radial energy and temperature profile, to better fit the specific area or dimensions or volume of the region-of-interest that is intended to be treated. For example, the same, single, waveguide and optical fiber, may propagate therein two (or more) different types of laser beams or light beams or light energy or laser-based energy, having two (or more, respectively) different wavelengths; and may cause those two (or more) beams to exit the waveguide and the optical fiber, sideways relative to the general direction of propagation (or, sideways relative to the long axis or long dimension of the waveguide or optical fiber); and may optionally divert or cause such two different types of laser beams or light beams to exit sideways at different locations and/or such that each one of them would enable medical treatment at a different in vivo location and/or at a different distance from the outer encapsulation of the optical fiber and/or at a different energy level.

For example, a first set of laser beams having a first wavelength λ1, may be diverted by a first optical element (or, by a first set of optical elements) within the waveguide or optical fiber, and may exit the optical fiber to super-impose at a first particular focal point (or focal region), located at a first distance D1 from the encapsulation sleeve of the optical fiber, and providing there a first level of energy E1; whereas, a second, different, set of laser beams having a second, different, wavelength 72, may be diverted by a second optical element (or, by a second set of optical elements) within the waveguide or optical fiber, and may exit the optical fiber to super-impose at a second, different, particular focal point (or focal region), located at a second distance D2 from the encapsulation sleeve of the optical fiber, and providing there a second level of energy E1; and in some embodiments, for example, D1 is different from D2, and/or E1 is different from E2. In some embodiments, the first wavelength λ1 is associated with a first level of absorption by human tissue or has a first absorption coefficient (A1); whereas the second wavelength λ2 is associated with a second, different, level of absorption by human tissue or has a second, different, absorption coefficient (A2).

In another example, a first set of laser beams having a first wavelength λ1, may be diverted by a particular optical element (or, by a particular set of optical elements) within the waveguide or optical fiber, and may exit the optical fiber to super-impose at a first particular focal point (or focal region), located at a first distance D1 from the encapsulation sleeve of the optical fiber, and providing there a first level of energy E1; whereas, a second, different, set of laser beams having a second, different, wavelength λ2, may be diverted by a that same particular optical element (or, by that same particular set of optical elements) within the waveguide or optical fiber, and may exit the optical fiber to super-impose at a second, different, particular focal point (or focal region), located at a second distance D2 from the encapsulation sleeve of the optical fiber, and providing there a second level of energy E1; and in some embodiments, for example, D1 is different from D2, and/or E1 is different from E2. In some embodiments, the first wavelength A1 is associated with a first level of absorption by human tissue or has a first absorption coefficient (A1); whereas the second wavelength λ2 is associated with a second, different, level of absorption by human tissue or has a second, different, absorption coefficient (A2).

In some embodiments, the fiber is embedded inside a structure such as a catheter or a guide-wire or a tube or a pipe or a probe, and a liquid media or a liquid medium surrounds the waveguide and/or the capsule around it. In accordance with some embodiments, the liquid medium may include a particular solution, or a mixture or combination of two or more solutions or liquids; and for some solutions, such as water with glucose or ethanol, the concentration of the solute changes the index of refraction. For example, for the solution of water solvent and glucose solute, an increase of the glucose concentration increases the index of refraction. Rays crossing an interface between two materials refract at angles which depend on the difference between the refraction indices of the material in the crossing. Changing the refraction index of one of the materials (e.g., the liquid medium that is surrounding the waveguide), leads to a change in the direction of the rays. In some embodiments where the rays are designed or structured to intersect at a distance from the waveguide, this change of refraction index would lead to an increase or to a decrease of the lateral distance of the focal point from the waveguide.

In some embodiments, another aspect or feature of the redistribution may be based on an apparatus or medical device allowing angular control over the laser-based energy emission. For example, the angular envelope is segmented to two or more sections. Each segment has a different optical element with a different emission profile. Each segment may have a different angular size and different energy density. The radial segments are spread along the longitudinal axis for the desired length, which may be different for each segment. The separation into segments and different power levels is useful for cases where the desired heating volume (or, the volume or the area of the region-of-interested that is intended to be treated) has a non-symmetrical structure, and/or the location of the probe is not exactly at the middle of the volume (or region) to be treated (e.g., due to spatial constraints or physical constraints which may block or limit or constrain the ability of the tip of the medical device from reaching or probing beyond a particular point, or due to a spatial obstacle, or due to a possible misalignment in the insertion or operation of the medical device).

In some embodiments, directionality of the emitted laser-based energy may be of importance in cases where adjacent to the volume (or region-of-interest) intended to be treated there is an area that should not be treated or that should be avoided from being exposed to the laser-based energy. For example, in certain brain treatments, there may be a functionally critical area (e.g., fornix, speech center, or the like) that is adjacent to (or neighboring, or in the vicinity of) a tumor that is intended to be treated; in treatment of prostate cancer, it is typically preferred not to treat the nearby anus area.

A demonstrative embodiment has one optical element with two angular segments: Segment A, with an angle of 90 degrees; and segment B, with an angle of 180 degrees. Segment A emits 90% of the source energy; Segment B emits 10% of the source energy. The density of the emission energy is controlled by the energy source and by the optical element structure; whereas the total power is a function of the energy source power, the optical element shape, and the total angular size. These are only non-limiting examples; some embodiments may provide a different number of such segments, which may be located at the same side or at different (opposing, or non-opposing) sides, and which may provide as output a different percentage of the source energy.

In some embodiments the optical element capability to control on the power and/or the direction of the laser-based or light-based energy may be a function of the relative area that the optical element covers, out of the entirety of the core cross-section of the waveguide or optical fiber.

In some embodiments, inwardly-facing or inwardly-directed wedges (or other suitable optical elements) may be structured as a continued or continues spiral shape or spiral structure, where the specific optical parameters are distributed in continuum over the spiral structure, depending on the radial location.

For demonstrative purposes, some of the drawings may depict a simplified version of actual ray tracing or a partial version thereof, in order to not obscure or over-crowd the drawings with multiple rays or beams or light routes.

For purposes of clarity, and to avoid over-crowding of the drawings, some of the drawings may depict a cross-section of a partial portion of a component (e.g., of a waveguide or a fiber optic or an optical fiber), and not the entirety of such component. It is to be understood that in such illustrations of a partial component, a similar structure may exist at the other portions or regions that are not shown in the drawing (e.g., continuing the same pattern that is depicted; or having a complementing or inverse or mirror-image structure thereof). Additionally or alternatively, the actual waveguide or fiber optic, or the medical device which incorporates such item, may include a series of the component(s) shown in a drawings, repeated in series or in a consecutive chain.

In some embodiments, a waveguide or an optical fiber may be constructed as a cylindrical or a generally-cylindrical elongated article, similar to a long and thin tube or pipe, and may be flexible or elastic or bendable or foldable. In some embodiments, optionally, a non-circular cross-section (e.g., oval or elliptical cross-section; egg-shaped cross-section) and/or a non-symmetrical cross-section may be used, for the entire medical device and/or for the sleeve or encapsulation element thereof, and/or for the waveguide itself and/or the optical fiber itself. It may transport and/or propagate and/or guide and/or direct laser-based energy, or laser beam(s), or light, or light-based energy, or optical energy, or light beam(s). It typically has a circular or generally-circular cross-section, or may have non-circular and/or non-symmetrical cross section as mentioned; and typically has a central Core that is surrounded by a (concentric, or generally concentric) Cladding or Clad. In some embodiments, the refractive index of the surrounding cladding, is slightly lower (e.g., by 0.5 or 1 or 1.5 or 2 percent) than the refractive index of the core. In some embodiments, the waveguide or optical fiber may be coated with a protective coating and/or a cushioning layer, and/or may be coated with (or may otherwise include) a rigid-flexible coating or layer, or a flexible or elastic coating or jacket or layer; and may optionally have one or more external marking(s) or other visual indicator(s) which may assist in the utilization of the medical device, particularly if the medical device is configured to emit energy in a non-symmetrical manner, or is configured to emit a first level of energy from a first particular side and to emit a second (different) level of energy from a second (different) side thereof.

In some embodiments, realized the Applicants, utilization of a non-circular (e.g., an oval or elliptical or egg-shaped cross section) and/or non-symmetrical cross-section, may provide one or more functional advantages; for example, it may provide to the user of the optical fiber (or the medical device) an increased mechanical control capability with regard to bending and/or flexing and/or moving the medical device in vivo, and/or it may provide a better indication to the user of the medical device with regard to which side of the optical fiber is facing which inner part of the body (e.g., and this may be particularly useful when the optical fiber emits, sideways, laser-based energy in accordance with a non-symmetrical or non-equal energy distribution scheme or energy emission scheme). In some embodiments, the non-circular cross-section may include, for example, at least one straight line (or straight surface), and at least one are, or curved or semi-circular line or partially-circular surface (or curved surface); for example, a cross-section that consists of a straight line, and an arc that encompasses less than 360 degrees (e.g., 330 or 300 or 270 or 180 degrees). Other suitable structures or cross-sections may be used.

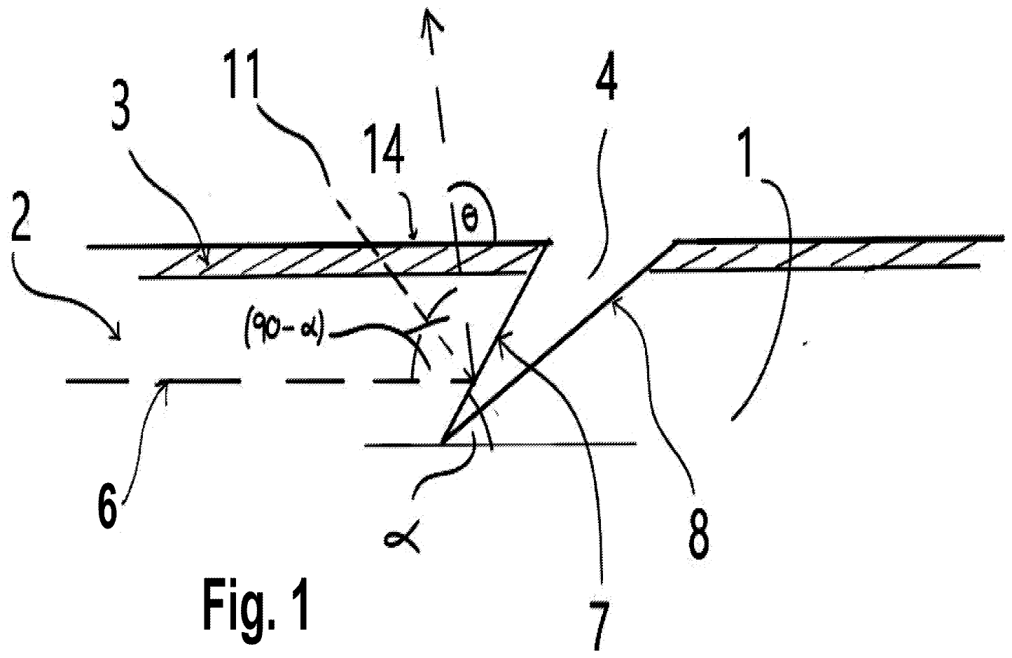

Reference is made to FIG. 1, which is a schematic illustration of a portion of a waveguide 1, particularly for propagating light or laser-based energy for medical treatment purposes, in accordance with some demonstrative embodiments. The view shown is of a longitudinal cross section, showing only half of the optical fiber for clarity purposes and in order to not over-crowd the drawing. A single optical component 4 is included, for demonstrative purposes; however, several or numerous such (or similar) optical components may be used in series. The light or the laser beam propagates inside waveguide 1, from left to right, through the core 2 that is surrounded by the clad 3. For example, a Ray 6 (e.g., light ray, light beam, laser beam) is coming from an energy source (e.g., a laser emitter, a laser transmitter, a laser generator, a light source, an optical energy source).

The internal structure of waveguide 1 includes an inward-facing protrusion or pin or wedge or tooth, defined in the drawing by planes (or surfaces) 7 and 8. Ray 6 reaches plane 7 which, is at an angle α relative to the main propagation direction of ray 6 (or, relative to the longest dimension of waveguide 1). Between plane 7 and plane 8 there is a gap with refraction index value that is lower than the refraction index value of the core 2, forming an inward-facing or inwardly-directed wedge or wedge-like or tooth-like structure or protrusion (or, forming a crater when viewed from externally to waveguide 1). Based on geometrical calculations, the incidence angle of ray 6 on surface 7 is (90°−α) in respect to vertical 11. As long as the value of (90−α) exceeds the critical angle, total inner reflection will reflect the ray back at the same angle according to Snell's law. The ray will eventually redirect to an angle θ, wherein θ=2*α, in respect to the initial propagation direction (e.g., this demonstrative calculation is for a ray that is parallel to the axis of propagation).

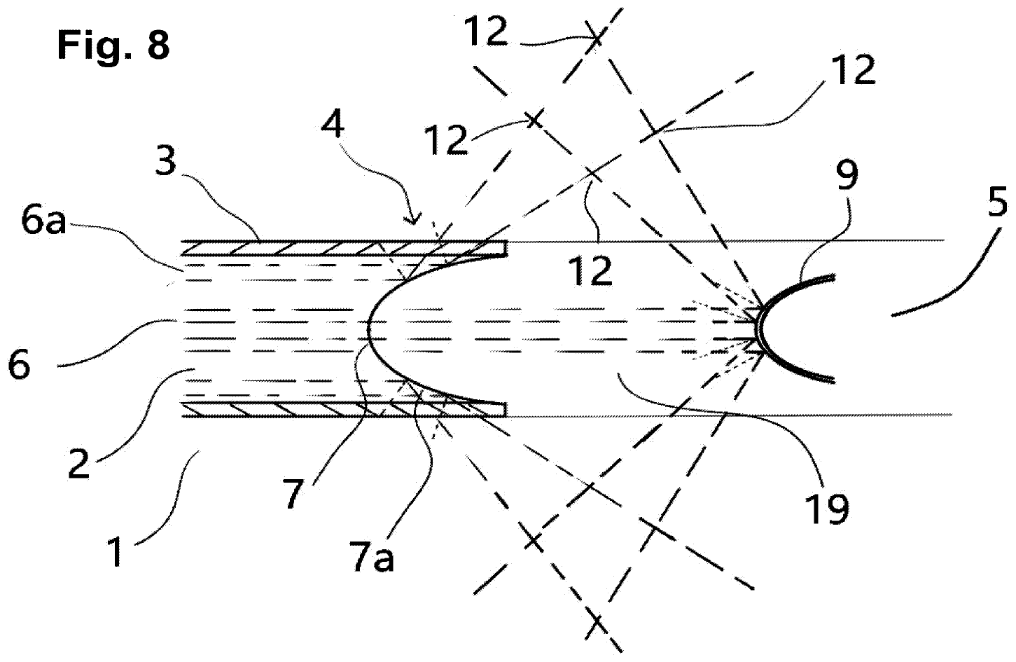

If the material outside the optical fiber has a different refraction index than the clad 3, then another redirection of the ray will occur upon reaching surface 14, according to Snell's law. For demonstrative purposes, and in order to avoid over-crowding of the drawings, the rays that are depicted are generally parallel to the long axis of the waveguide; however, these are only non-limiting examples, and some embodiments may similarly handle and utilize non-parallel rays, or rays that are not exactly parallel to the long axis of the waveguide; and in such cases, for example, instead of having a singular focal point externally to the waveguide, there may be a focal region or a focal zone externally to the waveguide (e.g., the quadrilateral or diamond-shaped area that is indicated by four numerals 12 in FIG. 8).

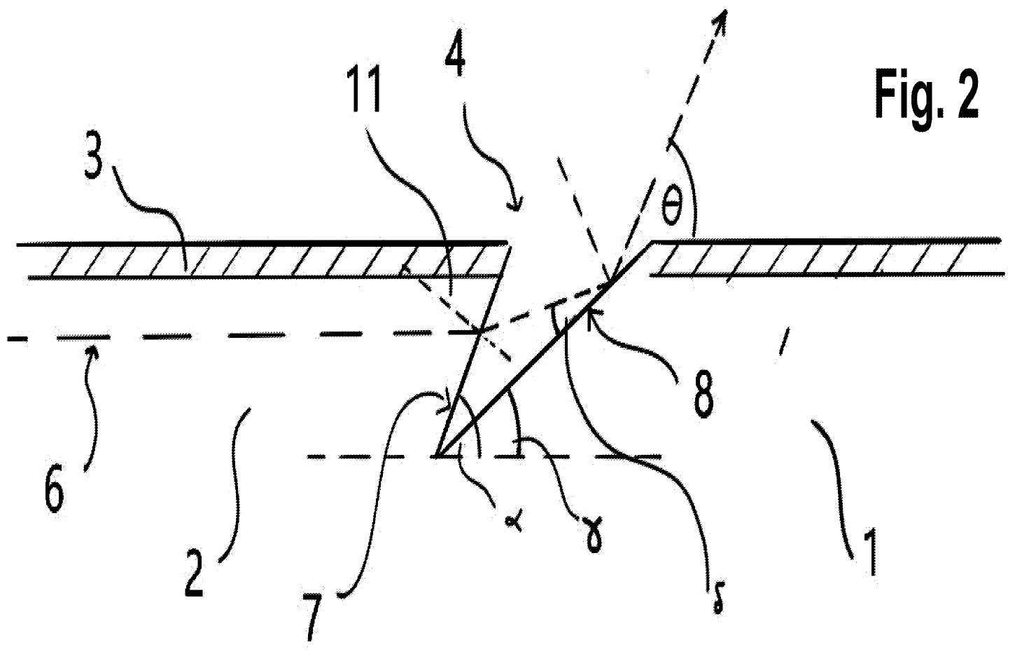

Reference is made to FIG. 2, which is a schematic illustration of another portion of waveguide 1, which is generally similar to the waveguide portion shown in FIG. 1, in accordance with some demonstrative embodiments. In FIG. 2, ray 6 reaches surface 7 at an angle of (90°−α), which does not exceed the critical angle in respect to vertical 11; and the ray will refract on surface 7 according to Snell's law, and continue to propagate towards surface 8, where it exhibits a specular reflection redirecting the ray an angle θ in respect to the original axis. The angle θ is the sum of: (i) the angle γ between surface 8 and the initial propagation direction, and (ii) the incidence angle δ of ray 6 on surface 8.

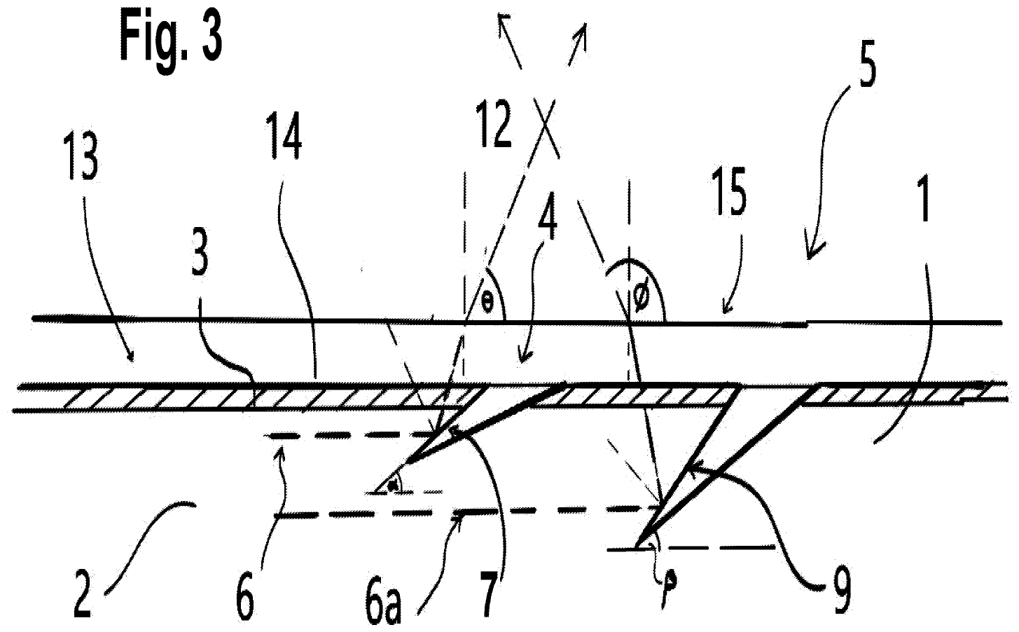

Reference is made to FIG. 3, which is a schematic illustration of a portion of waveguide 1, particularly for propagating light or laser-based energy for medical treatment purposes, in accordance with some demonstrative embodiments. It includes two non-identical optical elements 4 and 5, for example, two non-identical inwardly-facing wedges, having different penetration depth and/or different angle relative to the general direction of propagation. The two optical elements may be embedded in, or otherwise grooved into, a coating layer or coating member or encapsulating layer or a “jacket”, such as capsule 13. For demonstrative purposes, a non-limiting example may use the same or similar index of refraction for the capsule and clad materials; although some embodiments may utilize different values of index of refraction for the capsule and clad materials. The waveguide has a core 2 and a clad (or cladding) 3.

As demonstrated, two rays (6 and 6a) originate from a laser source on the left side and propagate to the right. Ray 6 reaches surface 7 of the first optical element 4 (the first inwardly-facing wedge), exhibits total internal reflection, and redirects at an angle of (2*α) relative to the long axis of the waveguide, reaching the outer surface 15 of capsule 13, and redirecting there again to an angle θ which is the sum of all reflections and the refraction upon reaching the media outside the waveguide. Ray 6a misses the first optical element 4, and continues to propagate through the long axis of the waveguide, reaching the second optical element 5 and its surface 9, where ray 6a reflects and redirects an angle (2*β) to the initial propagation axis, continuing to refract to a total angle Φ in respect to the original axis. In some embodiments, the rays reaching surface 15, move to a material with a lower refraction index than that of the capsule, and therefore the rays refract and increase the angle with respect to the vertical to surface 15. The waveguide is structured or configured such that Φ>θ, so the rays intersect outside the waveguide at point 12. The location and distance of point 12 from the waveguide is controlled or dictated by the parameters of the waveguide and the two optical elements; for example, based on their absolute dimensions, or based on their relative dimensions, or based on the ratio between the inwardly-facing depth of the first optical element 4 and the inwardly-facing depth of the second optical element 5, or based on the ratio between the inwardly-facing depth of the first optical element 4 and the diameter of the circular cross-section of the waveguide, or based on the ratio between the inwardly-facing depth of the second optical element 5 and the diameter of the circular cross-section of the waveguide. The rays (6 and 6a) continue to diverge after (or beyond) the focal point 12.

Accordingly, a High energy focal area (or, a laser-based High energy focal point; or a optical High energy focal point) is created or generated at or around point 12. This area or spatial region has higher energy density or concentration, due to the integration of the rays that intersect in it or in its immediate vicinity. The amount of superposition, and/or the scales of energy coming from each optical element (4 and 5), may be controlled or configured based on the specific properties of the optical unit; for example, by forming two particular optical elements (4 and 5) having specific vales of depth, slanting, three-dimensional shapes, longitudinal distance between them, or other parameters. This energy redistribution enables to change the temperature profile in the media or medium that the rays propagate in, from a simple exponential decay of energy, to a flatter radial profile.

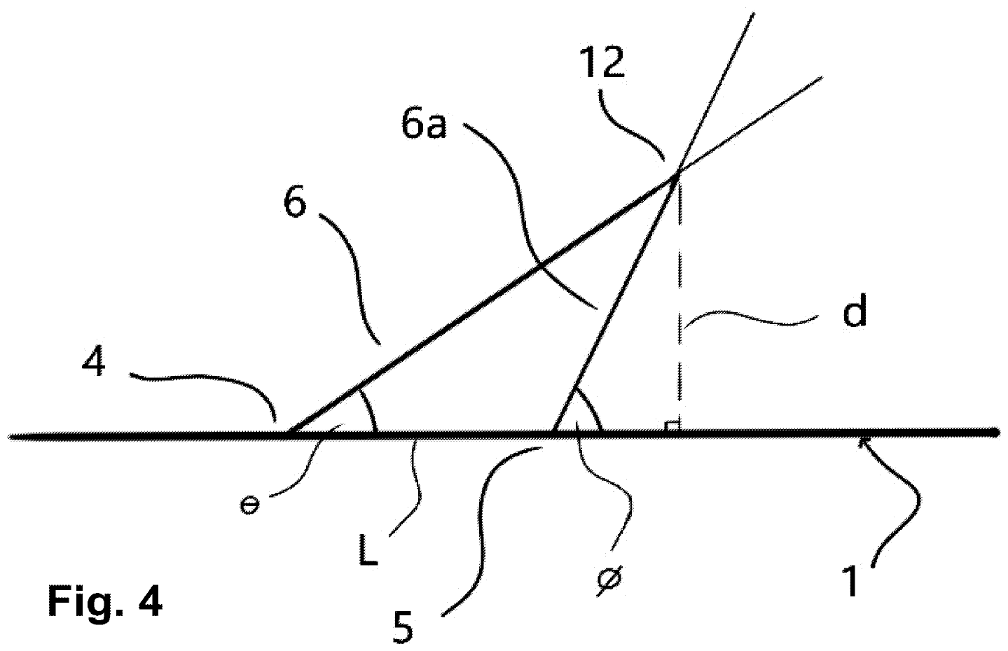

Reference is made to FIG. 4, which is a schematic illustration of a portion of waveguide 1, demonstrating schematically an optical view in accordance with some demonstrative embodiments. It demonstrates a schematic optical view of the optical unit, which may be similar to the optical unit discussed with reference to FIG. 3 above. The incoming ray 6 (or, a first portion of incoming rays) is partially reflected out of the waveguide by the first optical element 4, at an angle θ. Another incoming ray 6a (or, a second portion of the incoming rays) is emitted out of the second optical element 5, at an angle Φ. The angles are the overall product of all the internal refractions (including by the capsule itself). Optical element 5 is at a longitudinal distance L from optical element 4. The angle Φ is set to be larger than the angle θ, leading to the intersection of the rays (6 and 6a) at point, 12, at a vertical distance (d) from the main propagation axis. The distance (d) is dependent on the values of the angles and on the distance between the two optical elements. In some embodiments, the distance (d) may be calculated or estimated or approximated using the following demonstrative equation:

$$d = L sin θ sin Φ / sin ( Φ - θ )$$

Accordingly, some embodiments may control or configure or set the values of the geometric parameters, in order to tailor the energy profile to the volume of the region-of-interest that needs to be treated with light energy or laser-based energy or optical energy. In some embodiments, each medical device or each optical unit thereof may have a different value of distance (d), depending on the desired value of volumetric need.

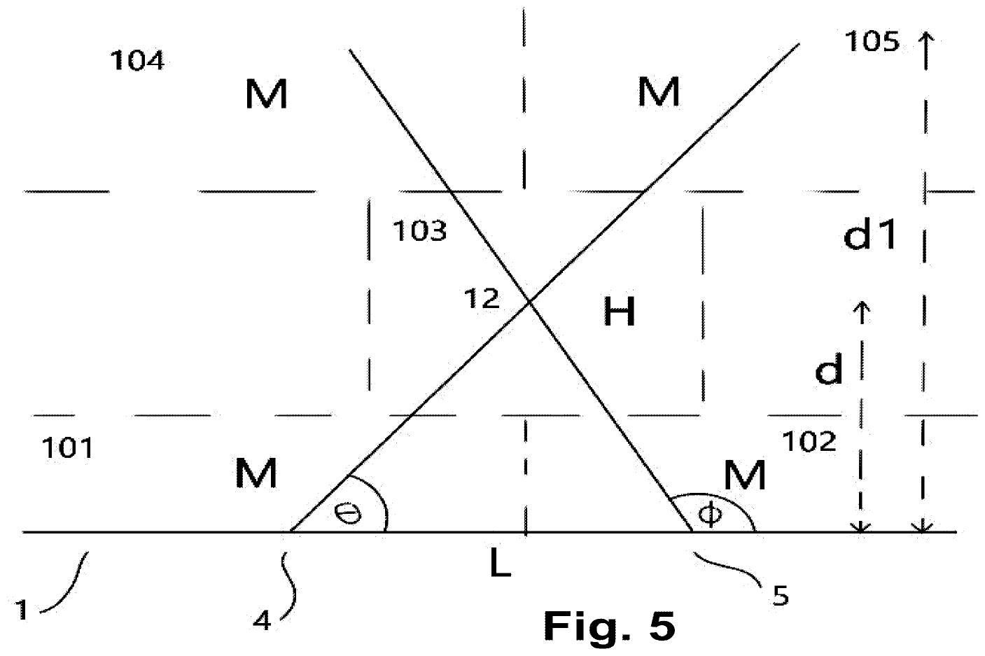

Reference is made to FIG. 5, which is a schematic illustration of a portion of waveguide 1, demonstrating schematically areas having different light-based energy profiles, in accordance with some demonstrative embodiments. It demonstrates an energy profile view of the effect of the optical unit on the adjacent media or medium. For demonstrative purposes, only two rays are shown, and only the upper side of the waveguide. The rays are emitted out of optical elements 4 and 5, at angles θ and Φ respectively. To clarify the energetic effect and consequentially the thermal effect, the area that is adjacent to (or is near) the waveguide 1 is divided into sections, indicated as generally rectangular areas in the drawing; each such section is marked with M for Medium or with H for High.

For example, Medium energy level is emitted from both optical elements at areas 101 and 102. The intersection of the rays at point 12 inside area 103 increases the energy to a High energy level at a distance d from the waveguide. Beyond it, the energy reduces again to a Medium level at areas 104 and 105. The result is a medium energy level area at a further distance, denoted d1, away from the waveguide (rather than adjacent to the waveguide). The distancing of the maximum energy level away from the waveguide may assist in the protection of the waveguide itself (e.g., by preventing cascading heating effects on the surface of the waveguide), in extending the mechanical and thermal resilience of the waveguide itself, and/or by extending of the reach of effective energy to a larger or further treatment volume or region-of-interest.

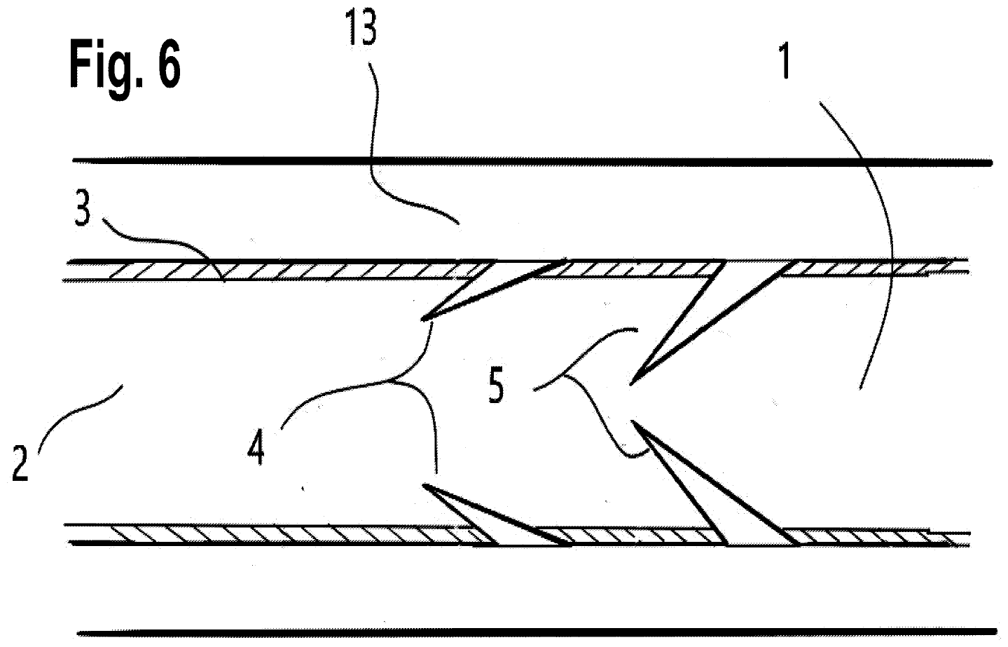

Reference is made to FIG. 6, which is a schematic illustration of a portion of waveguide 1, in accordance with some demonstrative embodiments. It shows a cross section of the waveguide portion, with the core 2 and the clad 3. An optical unit is constructed by two consecutive and non-identical optical elements (4 and 5), each one of them cover or encircling the full angular envelope. The waveguide may be within or inside capsule 13.

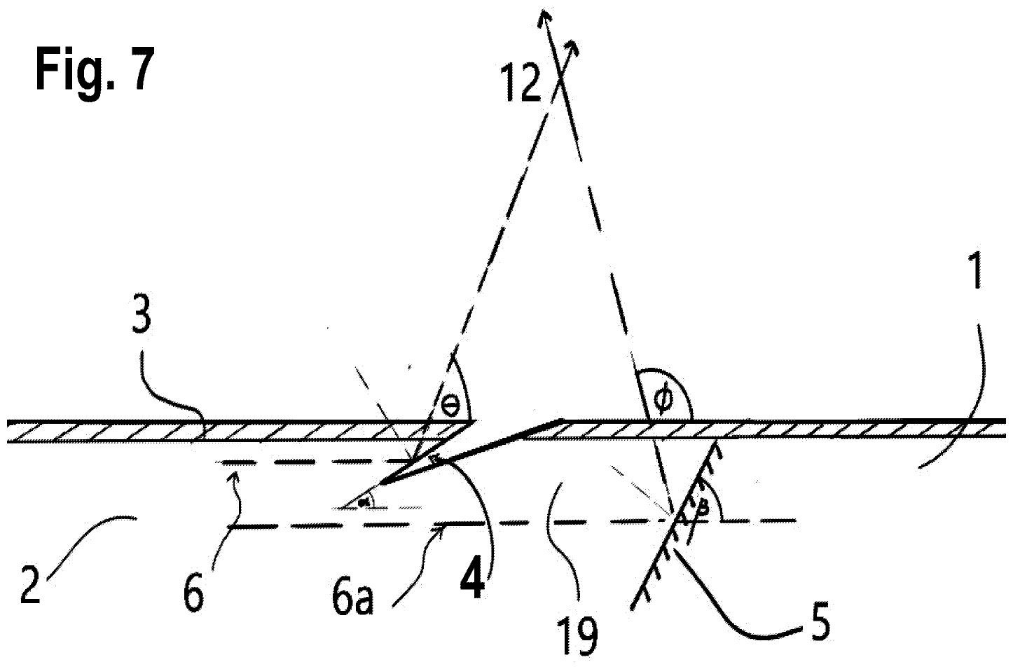

Reference is made to FIG. 7, which is a schematic illustration of a portion of waveguide 1, in accordance with some demonstrative embodiments. In this example, the optical unit has two optical elements (4 and 5); wherein optical element 5 is a mirror having specular reflection instead of total internal reflection. Rays 6 and 6a propagate from left to right. Ray 6 reflects at angle θ from optical element 4. Ray 6a reflects at angle Φ from optical element 5. Rays 6 and 6a converge at point 12, and then diverge beyond it. Area 19 is located between the two optical elements (4 and 5). Area 19 may be part of the core 2, or may be a void, or may be formed of a different material (from the material of the core 2) having a different refraction index.

Reference is made to FIG. 8, which is a schematic illustration of a portion of waveguide 1, in accordance with some demonstrative embodiments. It is a cross-sectional symmetric view of some embodiments that utilize one or more optical elements having curved surfaces. On the left there is shown a portion of waveguide 1, with its core 2 and clad (or cladding) 3. It ends with a curved surface 7, which has a part (denoted 7a) with a different incidence angle in relation to the axial ray. On the right there is shown a different, secondary, optical element 5 having a reflective surface 9. Rays 6 and 6a propagate from left to right, to optical element 4. Ray 6a reaches portion 7a of the curved surface of the optical element 4, at an angle which is larger than the critical angle (which is set by the interface between the material of core 2 and the material of area 19). Exceeding the critical angle yields reflection of ray 6a on surface 7a, at an angle which changes along the surface depending on its specific local curvature. Ray 6 propagates from left to right and reaches a different portion, portion 7, of the curved surface of optical element 4. Ray 6 meets that curved surface at an angle which is smaller than the critical angle, and therefore ray 6 proceeds to area 19 with minimal to no refraction (depending on the specific local curve of portion 7 of the curved surface of optical element 4). Upon reaching the curved surface 9 of the secondary optical element 5, some of the rays exhibit specular reflection at an angle in respect to the propagation direction, depending on the specific curvature. The curves of surface 7, 7a and 9 are set so the reflected rays from the surfaces would intersect at particular points 12 away from the waveguide, at several or at all possible combinations of the different reflections and refractions of the surfaces; for demonstrative purposes, eight such intersection points are illustrated, representing some of the possibilities).

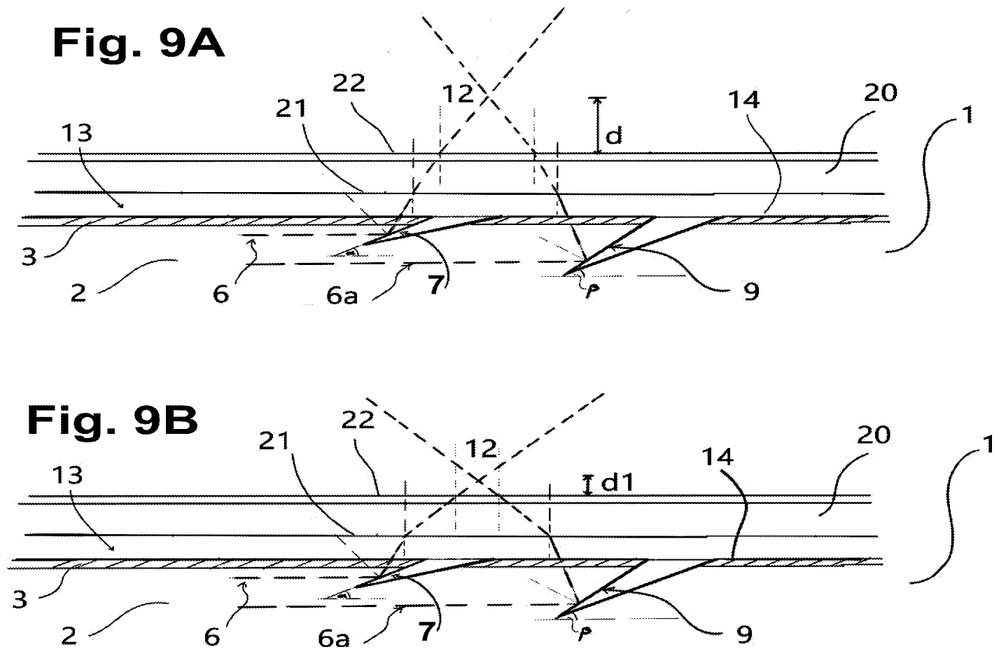

Reference is made to FIGS. 9A and 9B, which are schematic illustrations of a portion of waveguide 1, in accordance with some demonstrative embodiments; demonstrating modifications to the energy profile due to controlled changes in a controlled liquid or gas or fluid environment. The Applicants have realized that some liquids or gases or fluids or solutions may change their index of refraction, and/or their optical properties and/or the manner in which they allow laser beams or light or laser-based energy to propagate through them, in response to modification of an internal solvent and/or in response to modification or increase or decrease of a concentration of a solution, and/or in response to modification or increase or decrease of a ratio between ingredients or components of a solution or a mixture of materials. For example, a solution or a mixture of water with glucose (or with ethanol), may change its refractive index depending on the concentration of the glucose (or ethanol) in such solution or mixture. In accordance with some embodiments, the waveguide may be placed within a controlled (or configurable, or modifiable) liquid or gas or fluid or solution environment; such as, inside a catheter, such as within a liquid or gas or fluid or solution that surrounds the waveguide within the catheter (and which may also provide cooling). Accordingly, controlling or modifying the solvent concentration in the liquid or gas or fluid or the solution, may change the optical path of the rays redirected from the waveguide.

In some embodiments, for example, an external or ex vivo control unit may be controllably and/or selectively operated by an operator of the medical device, to dynamically modify or increase or decrease a concentration of a solution that is pumped or inserted or injected into a sleeve chamber or a sleeve storage layer, or into a fluid storage chamber or fluid holding chamber that is part of an encapsulation sleeve of the optical fiber, or into a fluid holding canal or a fluid holding channel that encapsulates at least a segment of the optical fiber, or into a corresponding chamber or channel or canal that may be an integral part of a catheter or of the medical device that comprises the optical fiber, or into a fluid-holding chamber or channel or canal or sleeve that is located in proximity or in immediate proximity to the outer side of the optical fiber and/or that directly touches the optical fiber and/or that is directly neighboring the optical fiber and/or that is directly attached to the optical fiber and/or that is directly attached within a catheter that also hosts the optical fiber.

It is noted that in some embodiments, the fluid-holding chamber or channel or canal or sleeve is implemented as a non-inflatable channel or canal or chamber or sleeve, which cannot be inflated by an inflow of air or gas, and/or which does not increase or modify its own volume in response to an inflow of air or gas, and/or which has fixed volume and/or fixed dimensions; such that the size or volume or contour of the medical device with such channel or canal or chamber or sleeve remains constant and does not change; and such that only the content of such channel or canal or chamber or sleeve may be changed, thereby changing the optical properties of such content but not changing the volume or size of the channel or canal or chamber or sleeve or the medical device. In other embodiments, optionally, the fluid-holding chamber or channel or canal or sleeve is implemented as an inflatable channel or canal or chamber or sleeve, such that also its size or volume or dimensions or contour may be modified by an operator, by changing the inflow of fluid or gas or liquid that is pumped or inserted or injected into such fluid-holding chamber or channel or canal or sleeve, and/or by performing inflation operations and/or deflation operations to achieve such changes.

In some embodiments, the control unit enables an operator to modify the concentration of the solution; to inject or insert or pump a solution into such fluid-holding chamber or sleeve or canal; to pump-out the content (or some of the content) from such fluid-holding chamber or sleeve or canal; to add only solids, or only liquids, into an already-inserted solution (e.g., in order to modify a concentration of a solution); to replace a first fluid or a first solution that was previously introduced into the fluid-holding chamber or sleeve or canal, with a second, different, fluid or solution having different optical properties; and/or to perform a combination of such modifications.

As demonstrated in FIG. 9A, waveguide 1 is located inside or within a catheter 22. Liquid 20 (or, in some embodiments, a gas, a mixture of gases, a fluid, a mixture of fluids, a solution) is in the volume between the capsule 13 and catheter 22. The overall direction of the rays in the media is the product of the optical element and the refraction on the two interfaces (21 and 22) where the rays move from one material to another. The changeable refractive index of the liquid may vary the overall direction of the rays. An advantage of such redirection of the rays is the ability to change the location of the focal point (12) of the rays, and/or its distance (d) from the waveguide, by changing the solution or its concentration.

In FIG. 9A, the index of refraction of the liquid (or gas, or fluid, or solution) is similar to that of the surrounding capsule; and therefore, the intersection of the rays is at focal point 12, located at distance d from the waveguide. FIG. 9B demonstrates a change in the location and/or distance of the focal point 12, upon modification of the index of refraction of the liquid or solution or gas or fluid (e.g., by modifying the concentration of the solvent in the solution). For example, the index of refraction of the surrounding liquid (or gas, or fluid, or solution) is reduced, and thus the rays shift to intersect at point 12 which is located at a reduced distance (d1) from the waveguide. For example, a controlled reduction in the index of refraction of the surrounding liquid (or gas, or fluid, or solution), may cause the focal point 12 to be located closer to the waveguide; and similarly, a controlled increase in the index of refraction of the surrounding liquid (or gas, or fluid, or solution), may cause the focal point 12 to be located further away from the waveguide.

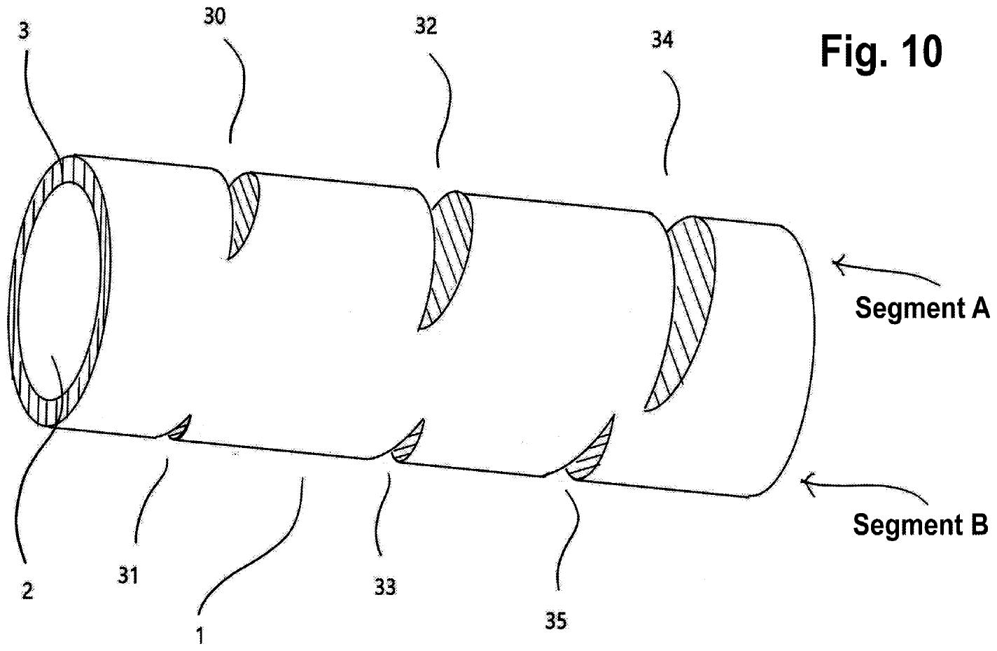

Reference is made to FIG. 10, which is an illustration of an isometric view of a portion of a waveguide with directional emission of the energy, in accordance with some demonstrative embodiments. It includes multiple inward-facing wedges, that are spread or placed or positioned at particular places or at particular longitudinal intervals along the longitudinal axis; and it provides an uneven energy emission. For example, waveguide 1 (or an optical fiber) having a core 2 and a clad (or cladding) 3, may have six optical elements embedded into it (denoted 30, 31, 32, 33, 34, and 35).

The group of six optical elements is divided to two segments on the angular envelope: optical elements 30 and 32 and 34 are in segment A, whereas optical elements 31 and 33 and 35 are in segment B. In this example, segment A is directing the energy emission in the upward direction of the drawing; whereas section B is directed the energy emission to the downward direction of the drawing. The direction of energy emission, the angular envelope, and the relative power of the energy emission are a product of (or are dependent on) the structure of the optical elements, their depth, their shapes, their length, the length between them, their slanting angle, and/or other configurable parameters.

In a demonstrative example, segment B has optical elements with smaller power density than the optical elements of segment A; while the power density values of each optical element in the same segment are similar to each other (e.g., P30=P32=P34>P31=P33=P35). The power density is controlled, or is configured or depends on, the amount of energy deflected from the core 2. The inwardly-facing wedges form optical elements that penetrate into the fiber core, to add portions of the fiber core that cause ray deflection. The inwardly-facing wedges are such that the increase in their penetration depth causes them to cover additional area within the optical fiber, and may be configured such that the same amount of energy would be emitted out of the core.

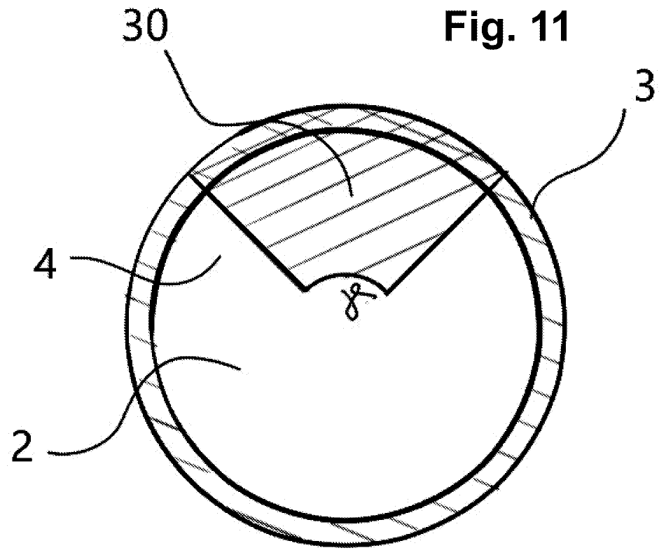

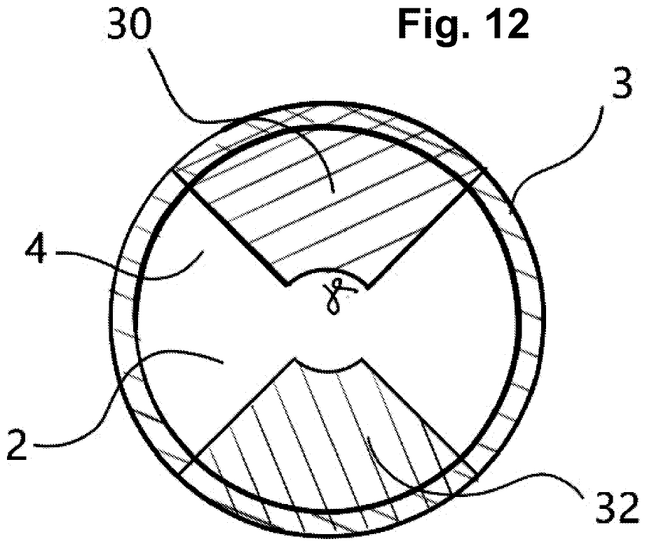

Reference is made to FIGS. 11-15, which are schematic illustrations of cross-sectional views of some waveguides, in accordance with some demonstrative embodiments. They demonstrate controlling the directional emission of energy of the fiber axis in different segments and in order to provide different power densities; enabling to control the direction, angle spread and also the relative power between the segments.

FIG. 11 shows a cross-sectional view of one optical element 4, which has a single segment 30 pointing upwards. Optical element 4 is shown with the core and the clad 3. In this example, the segment has an angle γ of 90°. The angle γ may vary as needed, for example, in the range of 10° to 180°. The areas that are not within the optical element 4 are the native optical fiber, or they may comprise one or more segments with different optical properties or optical functionalities. In the example shown, the optical element deflects (e.g., sideways relatively to, or away from, the long dimension of the optical fiber) approximately one quarter of the total laser-based or light-based energy.

FIG. 12 shows a cross-sectional view of two parallel optical elements: a top-side optical element 4 (pointing up, and corresponding to a top-side segment 30), and a bottom-side optical element 2 (pointing down, and corresponding to a bottom-side segment 32). In this case, each one of the two optical elements covers 90 degrees, and they have similar power density.

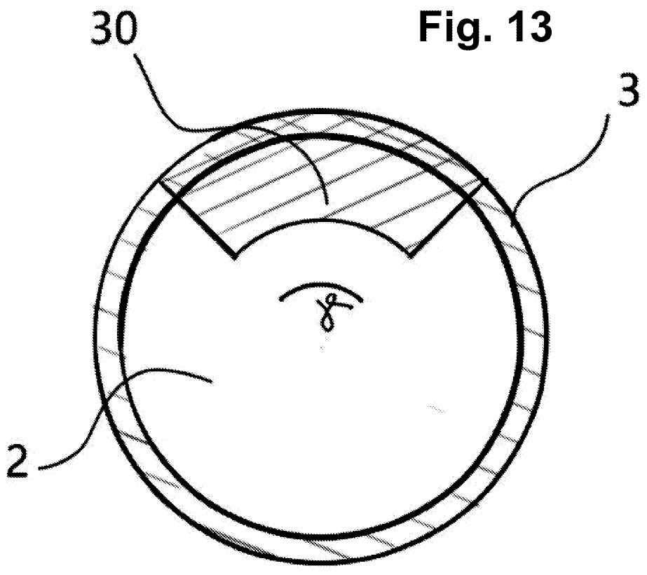

FIG. 13 shows a cross-sectional view of one optical element, which has a single segment 30 pointing upwards; it is generally similar in shape to the optical element shown in FIG. 11, but the optical element shown in FIG. 13 has a reduced area and thus provides a reduced power density relative to the power density provided by the optical element of FIG. 11. The reduced-area optical element of FIG. 13 thus deflects a smaller portion of the incident energy or the propagating rays. For example, due to its structure, the optical element of FIG. 13 deflects only the rays that reach (or that propagate near) the outer rim of the core; whereas rays that propagate at (or near) the center of the core would miss the deflection area of the optical element and would keep propagating beyond the optical element.

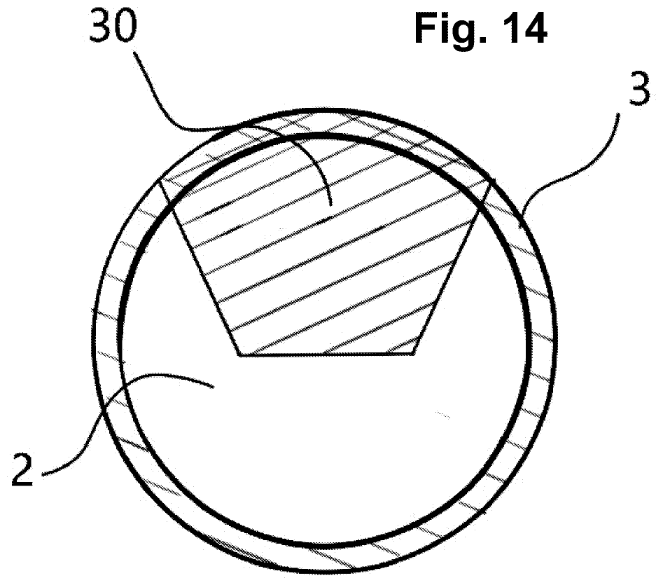

FIG. 14 shows a cross-section view demonstrating an optical element having a segment 30 providing an increased power density. The optical element provides a higher power density, due to the fact that a larger portion of the incident rays are deflected; even though the angular proportion of the segment is similar (e.g., 90 degrees). The deflection area is increased to redirect more energy from the main propagation axis. The direction of deflection depends mainly on the angle of the surfaces in the optical element, and thus remains the same.

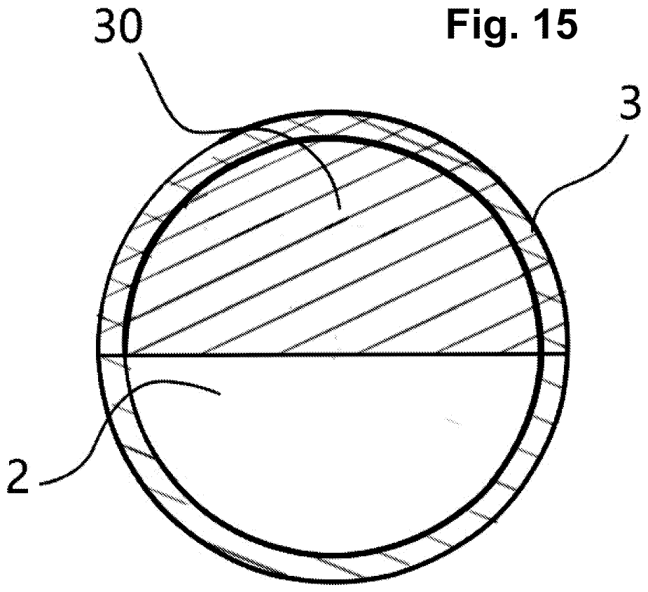

FIG. 15 shows a cross-section view demonstrating an optical element with a segment that covers 180 degrees, or half of the entire area of the cross-section of the optical fiber. Accordingly, half or approximately half of the propagating energy is deflected by the optical element.

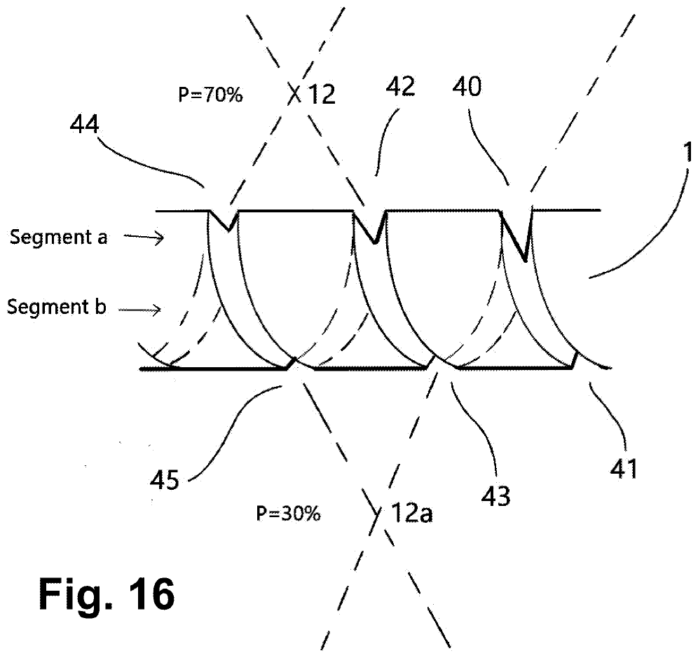

Reference is made to FIGS. 16-17 and 18A-18B, which are schematic illustrations of cross-sectional views of some additional waveguides, in accordance with some demonstrative embodiments. They demonstrate the integration of two or more of the features discussed above, in a single device.

In FIG. 16, waveguide 1 has a single continuous spiral or spiral-like optical element, that has different internal sections or different inwardly-facing regions or different inwardly-facing wedges, that combine or provide segmentation of some or all of the angular envelope, using regions that redirect the energy for focusing purposes. The continuous spiral has different segments; the direction of the optical element is configured or changed to meet the focusing requirements. The different segments characterize the direction and the power density of the emitted rays. For example, Segments 40, 42 and 44 provide an average power density that is higher than the average power density emitted from segments 41, 43 and 45. This arrangement or structure directs more laser-based energy or optical energy toward the upward direction, relative to the downward direction. The weight or the ratio of the relative power that is emitted, may be adjustable or configurable for different directions, and/or may be different within regions of the same segment (if desired, to achieve particular implementation goals). The direction of the emitted rays is also controlled by the direction and/or size and/or shape and/or slanting of the optical elements in each segment and/or the planes or surfaces that are used for constructing each segment or each element (e.g., each wedge). As a non-limiting example, segment 44 directs the emitted energy in diagonal to the right (north-east), whereas the same-side neighboring segment 42 directs the emitted energy to the left (north-west); and the rays emitted from those two segments (42 and 44) intersect at point 12 above the waveguide. Similarly, segment 45 directs the emitted energy in diagonal to the right (south-east), whereas the same-side neighboring segment 43 directs the emitted energy to the left (south-west); and the rays emitted from those two segments (45 and 43) intersect at point 12a below the waveguide. In a demonstrative example, the density of the combined power at focal point 12 (or, near that focal point 12; or, in all the focal points that are located upwardly relative to the waveguide) is 70% of the total power that initially propagated into the waveguide; and the density of the combined power at focal point 12a (or, near that focal point 12a; or, in all the focal points that are located downwardly relative to the waveguide) is 30% of the total power that initially propagated into the waveguide.

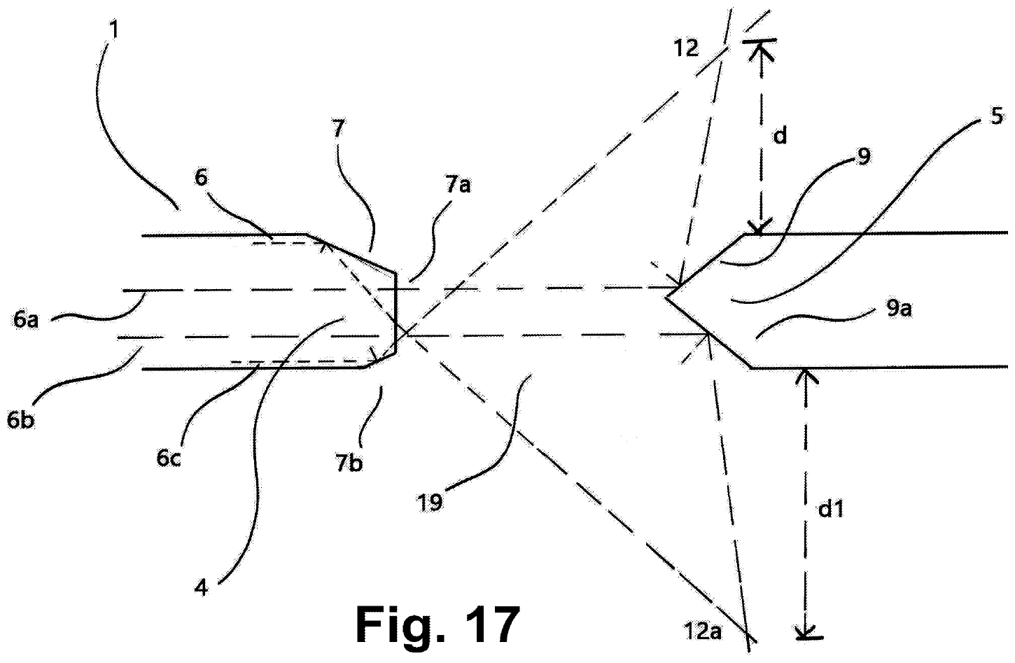

In FIG. 17, there is depicted an optical unit that may be utilized to achieve energetic directionality, for example, in parallel to focal extension of the energy. For example, such structure may direct a first particular percentage of the energy towards the downward direction, and another, different, particular percentage of the energy towards the upward direction; while creating two different focal points, one focal point located on each side of the waveguide, at distances d and d1 (respectively) from the waveguide. The waveguide has two optical elements: a first optical element 4 (e.g., having three surfaces: 7, 7a, 7b), and a second optical element 5 (e.g., having two surfaces: 9 and 9a). Optical element 5 is reflective, for example, by coating with a reflecting material, or by other suitable means. Rays 6, 6a, 6b and 6c are propagating in the waveguide, from left to right. Rays 6 and 6c are reflected by optical element 4; while rays 6a and 6b refract on surface 7a, and propagate towards the second optical element 5 where they are redirected. Ray 6 is directed towards surface 7a, and goes through total internal reflection and redirects to the downward side. Ray 6a and ray 6b pass through surface 7a and area 19, and reach optical element 5. Ray 6a reflects on surface 9 and redirects upwardly. Ray 6b reflects on surface 9a and redirects downwardly. Ray 6c reaches surface 7b and goes through total internal reflection, and redirects upwardly. Ray 6a and ray 6c intersect at upper focal point 12, located at distance d upwardly from the waveguide. Ray 6 and ray 6b intersect at lower focal point 12a, located at distance d1 downwardly from the waveguide. The area of surface 7 is larger than (e.g., 2 or 2.5 or 3 times greater than) the area of surface 7b. The ratio of the cross-sectional surface areas, or surface 7 and 7a, are configured or set such that the redirected energy would maintain a desired ratio of emitted energy towards the two (or more) directions; for example, in this case, a ratio of 70% to 30% for surfaces 7-7b and 9-9a (excluding the shadows of areas 7 and 7b, respectively).

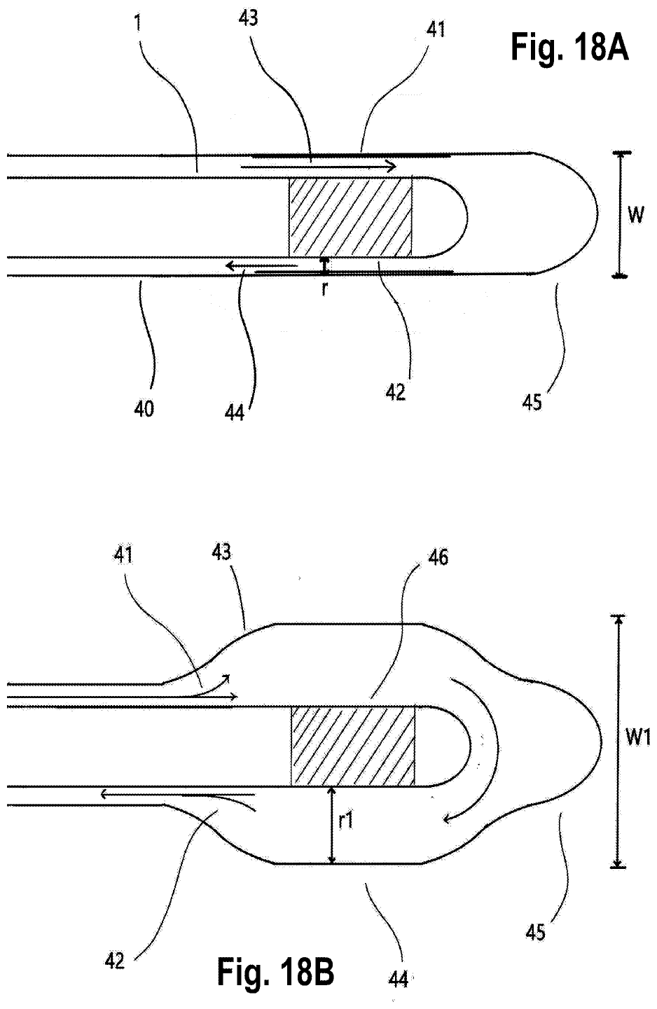

Reference is made to FIGS. 18A and 18B, which are illustrations of a portion of a waveguide or optical fiber, enclosed in (or surrounded by) an inflatable balloon, in accordance with some embodiments. For example, a tip of a catheter is constructed from a stretchable material or elastic material or inflatable material, that can be inflated or expanded or shrunk by adding (or removing) a proper pressure, to thereby selectively increase (or decrease) the volume of the inflated balloon that surrounds (or that is near) the catheter tip, particularly at the emission zone of the optical fiber. The balloon may be, for example, a compliant balloon or elastomeric balloon (e.g., formed of polyurethane or silicone), inflated by volume (rather than by pressure); or a non-compliant balloon or high-pressure balloon (e.g., formed of polyester or nylon); or a semi-compliant balloon or mid-pressure balloon.

The balloon may cover the azimuthal angle of the catheter; or only a portion or part thereof. An advantage of increasing the volume of the balloon around the catheter tip may be, in some embodiments, an enhancement of the cooling of the optical fiber area, and/or a decrease in the energy concentration at the tissue that is immediately adjacent to the catheter tip itself; which in turn may help prevent overheating of the tissue, and/or may protect the optical fiber itself from getting damaged due to overheating; and/or a distancing of the encapsulating medium, which may in turn cause modification of the direction of the emitted energy and/or the distance (from the waveguide's outer jacket) in which High energy focal points (or focal zones, or focal regions) occur.

In FIG. 18A, the balloon is shown in non-inflated state, or in idle state, or in deflated state. In FIG. 18B, the balloon is shown in inflated state. For example, as demonstrated in FIG. 18A, the optical fiber 1 is inserted into the catheter 40. An area 41 of the catheter is an area structured by an expandable material, forming a balloon like region in the catheter. The light-emitting zone 42 of the optical fiber is placed or is located near or before the catheter tip 45 (e.g., and not at or within the catheter tip itself), and/or is adjacent to area 41 which is the balloon area of the catheter 40. The space or gap or pocket or channel between the catheter 40 and the optical fiber is flushed with fluid (e.g., saline), entering from area 43 and exiting from area 44, which can be used for cooling and/or for inflating (or deflating) the balloon. As long as the fluid pressure within the balloon is low, the balloon stays deflated and the distance (r) between the optical fiber and the catheter is similar or identical in this area relative to other areas along the catheter. The maximal outer diameter of the catheter is denoted as w.

In FIG. 18B, the fluid is pressurized, flowing inside the balloon via a pathway 43 and exiting the balloon via a pathway 44, while the pressure drop between the inflow and outflow causes the balloon to inflate. In this demonstrative example, the balloon is non-compliant and the balloon reaches its full predetermined size as the gap between the optical fiber and the catheter increases from r to r1 at area 41 of the catheter (namely, r1>r). The maximal diameter of the catheter increases from w to w1 (namely, w1>w). This effectively increases the gap between the optical fiber and the tissue, with the fluid acting as a buffer between them at the area of energy emission of the optical fiber. This increase causes the emitted energy to spread over an area which is effectively larger by the proportion ˜r1/r, thus reducing the density of emitted energy at the first contact with the tissue by the same (or similar) proportion, and as a result reduces the risk of a hot spot or a heat burn in the contact area. Tissue compression around the balloon also increases the effective diameter of the applied energy.

Some embodiments may include a medical device or a catheter or a medical probing device or a medical treatment device, having therein an optical fiber or a waveguide or optical unit able to direct and propagate and emit laser-based energy or light-based energy or optical energy. The optical unit comprises two (or more) optical elements in the waveguide. For example, a proximal element A emits a first portion of the energy (energy portion I) in an angle θ relative to the original direction of propagation. A distal element (or a following element, or a secondary element, or a consecutive element) B emits a second portion of the energy (energy portion II) in another angle Φ relative to the original direction of propagation. Angles θ and Φ are configured or set, such that the rays intersect at a particular distance (d) from the waveguide axis, and diverge from that point on. Each optical unit may have more than two optical elements, that may intersect (externally to the waveguide) at a single focal point or at multiple focal points. Each waveguide may include more than one optical unit. In some embodiments, consecutive optical units (or, optical units connected in series) may emit different fractions of the entirety of rays that entered into the core of the waveguide.

In some embodiments, the optical unit has two or more optical elements; and each optical element redirects a portion of the total energy of the waveguide at a particular different slanting angle relative to the original direction of propagation. The angular envelope of the waveguide or optical fiber is segmented to two or more different segments; each such segment providing a different energy emission profile.

In some embodiments, the optical unit (or at least a portion thereof) is located within a liquid or a solution or a fluid (e.g., stored or held within a sleeve or chamber or channel or canal, such as a fluid-storing canal or channel that encapsulates or encircles or coats or encloses at least a segment of the optical unit or optical fiber or waveguide), and the refractive index of such liquid or solution or fluid may be modified or configured or controlled, thereby affecting the distance (d) by changing properties of the liquid or the solution in real time or near real time (e.g., by changing or replacing the fluid; by changing the concentration or clarity or transparency or translucency of brightness level or contrast level of the fluid; by adding solvent into a solution; or the like).

In some embodiments, the optical element may be, for example, an inwardly-facing wedge or tip or reflecting element or refractive element, or an inwardly-facing protrusion or rib or prism, or an inwardly-facing slanted optical hurdle, or an inwardly-facing object having one or more curved surfaces and/or one or more planar surfaces, or other type of prism or lens or curved mirror or convex mirror or concave mirror or planar mirror.

Some embodiments provide a medical device, comprising: an optical fiber configured to be inserted, at least partially, into a body of a human; wherein the optical fiber is flexible, and has a proximal end that remains outside the body of the human, and a distal end that is controllably movable within the human body by one or more operations of a medical device operator that are selected from the group consisting of: pushing, pulling, bending, spinning, turning, flexing. The optical fiber is configured to receive, at said proximal end, one or more laser beams that are generated by a laser beam generator that is operably associated with said optical fiber. At least one region or segment of the optical fiber includes, at an internal side of said optical fiber, at least one optical element selected from the group consisting of: (i) a refracting optical element that refracts one or more laser beams that propagate within the optical fiber, (ii) a deflecting optical element that deflects one or more laser beams that propagate within the optical fiber, (iii) an optical element that deflects one or more laser beams that propagate within the optical fiber and refracts one or more other laser beams that propagate within the optical fiber. The at least one optical element, that is located at said internal side of said optical fiber, performs deflecting and/or refracting of one or more laser beams that propagate through said optical fiber in accordance with a particular laser-energy distribution and emission scheme, and directs at least one laser beam to exit said optical fiber sideways relative to a long axis of said optical fiber through a side-wall of the optical fiber and to provide laser energy to an in-vivo location that is located sideways relative to said optical fiber.

In some embodiments, the at least one optical element, that is located at said internal side of said optical fiber, is configured to direct two or more laser beams to exit sideways relative to the long axis of said optical fiber, and to intersect and super-impose at a particular distance sideways relative to the long axis of said optical fiber.

In some embodiments, the at least one optical element, that is located at said internal side of said optical fiber, is configured to direct one or more laser beams to exit sideways from said optical fiber towards a first direction, and is configured to direct one or more other laser beams to exit sideways from said optical fiber towards a second, different, direction.

In some embodiments, the at least one optical element comprises: (I) a first optical element, that is located at a first location of the internal side of the optical fiber, and is configured to direct one or more laser beams to exit sideways from said optical fiber towards a first direction; and also, (II) a second optical element, that is located at a second, different, location of the internal side of the optical fiber, and is configured to direct one or more laser beams to exit sideways from said optical fiber towards a second, different, direction.

In some embodiments, the at least one optical element comprises: (I) a first optical element, that is located at a first location of the internal side of the optical fiber, and is configured to direct two or more laser beams to exit sideways from said optical fiber towards a first direction, and to intersect and super-impose at a first intersection region that is located at a first distance from said optical fiber; and also, (II) a second optical element, that is located at a second, different, location of the internal side of the optical fiber, and is configured to direct two or more laser beams to exit sideways from said optical fiber towards a second, different, direction, and to intersect and super-impose at a second, different, intersection region that is located at a second, different, distance from said optical fiber.

In some embodiments, the at least one optical element comprises: (I) a first optical element, that is located at a first location of the internal side of the optical fiber, and is configured (i) to direct two or more laser beams to exit sideways from said optical fiber towards a first direction, and to intersect and super-impose at a first intersection region that is located at a first distance from said optical fiber; and (ii) to output sideways towards said first direction N percent of an entirety of laser energy that entered the optical fiber via the proximal end; and also, (II) a second optical element, that is located at a second, different, location of the internal side of the optical fiber, and is configured (i) to direct two or more laser beams to exit sideways from said optical fiber towards a second, different, direction, and to intersect and super-impose at a second, different, intersection region that is located at a second, different, distance from said optical fiber, and (ii) to output sideways towards said second direction M percent of an entirety of laser energy that entered the optical fiber via the proximal end; wherein N is smaller than 100, and wherein M is smaller than 100. In some embodiments, N is different than M. In some embodiments, N is not necessarily different than M.

In some embodiments, the distal end of the optical fiber comprises a cap element or a tip element; a laser energy E enters said optical fiber via the proximal end; the at least one optical element, located at the internal side of the optical fiber, is configured to divert N1 percent of said laser energy to exit sideways relative to said optical fiber; wherein N2 percent of said laser energy is routed by said optical fiber and exits said optical fiber at the distal end, via said cap element or tip element, in a forward direction and not in a sideway direction relative to the optical fiber. In some embodiments, N1 is smaller than 100, and N2 is smaller than 100. In some embodiments, optionally, N1 is different than N2.

In some embodiments, the distal end of the optical fiber comprises a cap element or a tip element; wherein a laser energy E enters said optical fiber via the proximal end; wherein the at least one optical element comprises a first optical element and a second optical element, that are located at two different locations within the optical fiber. The first optical element within the optical fiber, is configured to divert N1 percent of said laser energy to exit sideways relative to said optical fiber, towards a first sideways direction. The second optical element within the optical fiber, is configured to divert N2 percent of said laser energy to exit sideways relative to said optical fiber, towards a second, different, sideways direction. Furthermore, N3 percent of said laser energy is routed by said optical fiber and exits said optical fiber at the distal end, via said cap element or tip element, in a forward direction and not in a sideway direction relative to the optical fiber. In some embodiments, N1 is smaller than 100; and N2 is smaller than 100; and N3 is smaller than 100. In some embodiments, N1 is different than N2.

In some embodiments, the at least one optical element comprises at least: an inwardly-facing wedge or an inwardly-facing protrusion, that diverts at least some of laser beams that enter the optical fiber, to exit the optical fiber sideways and not through the distal end of the optical fiber.

In some embodiments, the at least one optical element comprises at least: (I) a first inwardly-facing slanted wedge, that diverts a first portion of laser energy that enters the optical fiber, to exit the optical fiber sideways towards a first sideways direction relative to the optical fiber, and not through the distal end of the optical fiber; and also, (II) a second inwardly-facing slanted wedge, that diverts a second, different, portion of laser energy that enters the optical fiber, to exit the optical fiber sideways towards a second, different, sideways direction relative to the optical fiber, and not through the distal end of the optical fiber.

In some embodiments, the at least one optical element comprises at least: an inwardly-facing, generally spiral or generally helical, elongated protrusion, that spirals internally within the optical fiber as an internal spiral protrusion or as an internal helical protrusion, and that provides at least one of: (i) a continuum of modification of optical properties of laser energy that propagates within the optical fiber, (ii) a continuum of guidance of laser beams that propagate internally within the optical fiber; and that diverts at least some of laser beams that enter the optical fiber, to exit the optical fiber sideways and not through the distal end of the optical fiber.

In some embodiments, the at least one optical element comprises exactly one optical element, or comprises two or more optical elements; that are located internally within the optical fiber, and that divert laser energy to exit non-symmetrically and sideways from said optical fiber. A first portion of laser energy that enters the optical fiber via the proximal end, exits sideways from said optical fiber, not via the distal end of the optical fiber, and towards a first sideways direction. A second, different, portion of laser energy that enters the optical fiber via the proximal end, exits sideways from said optical fiber, not via the distal end of the optical fiber, and towards a second, different, sideways direction that is non-symmetrical relative to said first sideways direction.

In some embodiments, the at least one optical element is located internally within the optical fiber, and diverts a first portion of laser energy that entered the optical fiber via the proximal end, to exit the optical fiber sideways and to enable medical treatment via laser energy via a side-wall of the optical fiber and not via the distal end of the optical fiber. The at least one optical element also diverts a second portion of laser energy, that entered the optical fiber via the proximal end, to exit the optical fiber in a forward direction via the distal end of the optical fiber.

In some embodiments, the medical device further comprises: an encapsulation sleeve, that encapsulates at least a segment of the optical fiber; wherein said segment of the optical fiber is intended to be inserted into the body of said human. The encapsulation sleeve comprises a non-inflatable fluid-holding canal, that is configured to receive an inflow of a fluid that causes a modification of a focal point at which two or more laser beams intersect and super-impose after they exit sideways via a wall of said optic fiber and via said fluid-holding canal. Said fluid comprises one or more of: a gas, a liquid, a solution, a saline solution, a solution of water and sugar, a solution of water and salt.

In some embodiments, the medical device further comprises: a control unit that is connected ex vivo to the optical fiber, and that selectively or temporarily pumps a particular fluid into said non-inflatable fluid-holding canal or out of said non-inflatable fluid-holding canal; and selective or temporarily causes a modification of the focal point at which two or more laser beams intersect and super-impose after they exit sideways via the wall of said optic fiber and via said non-inflatable fluid-holding canal.

In some embodiments, the medical device further comprises: a control unit that is connected ex vivo to the optical fiber, and that selectively or temporarily pumps a particular material into said non-inflatable fluid-holding canal or out of said non-inflatable fluid-holding canal, and thus modifies a concentration of a solution that is held within said non-inflatable fluid-holding canal; and selective or temporarily causes a modification of the focal point at which two or more laser beams intersect and super-impose after they exit sideways via the wall of said optic fiber and via said non-inflatable fluid-holding canal.

In some embodiments, the medical device further comprises: an encapsulation sleeve, that encapsulates at least a segment of the optical fiber; wherein said segment of the optical fiber is intended to be inserted into the body of said human; wherein the encapsulation sleeve comprises, at said segment, an inflatable balloon chamber, that is controllably inflatable in vivo by receiving an inflow of a fluid that causes inflation of said inflatable balloon chamber and thus causes a modification of a distance between (i) an inner wall of the optical fiber and (ii) an in vivo point-of-interest to be medically treated, and in turn causes a modification of a power density of laser-based energy that reaches said in vivo point-of-interest; wherein said fluid comprises one or more of: a gas, a liquid, a solution, a saline solution, a solution of water and sugar, a solution of water and salt.

In some embodiments, the at least one optical element comprises at least: (a) a first curved optical element, that (a1) diverts a first portion of laser energy that enters the optical fiber, to exit the optical fiber sideways towards a first sideways direction relative to the optical fiber, and not through the distal end of the optical fiber; and that (a2) allows a second portion of the laser energy that enters the optical fiber, to proceed with non-modified propagation towards a second, different, curved optical element that is located further along said optical fiber; (b) said second, different, curved optical element, that is located further along said optical fiber, and that receives said second portion of the laser energy, and that diverts said second portion of laser energy to exit the optical fiber sideways towards a second, different, sideways direction relative to the optical fiber, and not through the distal end of the optical fiber; wherein the first portion of laser energy that exited sideways from the optical fiber due to the first curved optical element, and the second portion of laser energy that exited sideways from the optical fiber due to the second curved optical element, collide and super-impose at an in vivo region-of-interest that is a focal region and is not a singular focal point.

In some embodiments, the optical fiber has a non-circular or non-symmetrical cross-section, (i) which improves mechanical control of bending operations performed by an operator of the medical device, (ii) and which enables the operator of the medical device efficient understanding of the in-vivo spatial orientation of the optical fiber.

In some embodiments, the optical fiber has an oval or egg-shaped cross-section.

In some embodiments, the optical fiber has a cross section that consists of: a straight line, and an arc of less than 300 degrees (or, an arc or less than 330 degrees; or, an arc of less than 270 degrees).

In some embodiments, the optical fiber is configured, due to incorporation of said at least one optical element therein, to emit directional, sideways, non-forward directed, laser energy; which is emitted sideways through a side-wall of the optical fiber and not through a cap or tip located at the distal end of the optical fiber; in accordance with a weighted energy emission scheme that defines: (i) that N1 percent of laser energy that entered the optical fiber via the proximal end is emitted sideways through a first location of a side-wall of the optical fiber towards a first sideways and non-forward direction, and (ii) that N2 percent of laser energy that entered the optical fiber via the proximal end is emitted sideways through a second location of the side-wall of the optical fiber towards a second sideways and non-forward direction, and (iii) that N3 percent of laser energy that entered the optical fiber via the proximal end is emitted in a forward direction relative to the optical fiber and via the cap or tip of the distal end of the optical fiber. In some embodiments, N1 is different from N2 and is different from N3; and also, N2 is different from N1 and is different from N3; and also, N3 is different from N1 and is different from N2.

In some embodiments, the optical fiber is configured, due to incorporation of said at least one optical element therein, to emit directional, sideways, non-forward directed, laser energy; that performs a medical procedure at a body-location that is located sideways relative to a longest dimension of the optical fiber, and that is not located at a forward direction relative to the longest dimension of the optical fiber; and that provides laser energy to said body-location that is located sideways relative to the longest dimension of the optical fiber in accordance with a pre-defined or dynamically-modifiable energy distribution scheme.

In some embodiments, the optical fiber is configured to receive, at the proximal end of the optical fiber, from said laser beam generator that is operably associated with said optical fiber: (I) a first set of laser beams having a first wavelength λ1, having a first absorption coefficient A1 indicating a level of absorption by human body tissue, wherein the first set of laser beams having the first wavelength λ1 are diverted within the optical fiber by a first set of optical elements, which cause the first set of laser beams having the first wavelength λ1 to emit sideways from the optical fiber and to super-impose at a first particular focal point or focal region that is located at a first distance D1 from an outer layer of the optical fiber, wherein the first set of laser beams having the first wavelength λ1 provides a first level of energy E1 at said first particular focal point or focal region that is located at said first distance D1; and (II) a second, different, set of laser beams having a second, different, wavelength λ2, having a second, different, absorption coefficient A2 indicating the level of absorption by human body tissue, wherein the second set of laser beams having the second wavelength λ2 are diverted within the optical fiber by a second set of optical elements, which cause the second set of laser beams having the second wavelength λ2 to emit sideways from the optical fiber and to super-impose at a second, different, particular focal point or focal region that is located at a second, different, distance D2 from the outer layer of the optical fiber, wherein the second set of laser beams having the second wavelength λ2 provides a second, different, level of energy E2 at said second particular focal point or focal region that is located at said second distance D2.

In some embodiments, the optical fiber is configured to receive, at the proximal end of the optical fiber, from said laser beam generator that is operably associated with said optical fiber: (I) a first set of laser beams having a first wavelength λ1, having a first absorption coefficient A1 indicating a level of absorption by human body tissue, wherein the first set of laser beams having the first wavelength λ1 are diverted within the optical fiber by a particular optical element, which causes the first set of laser beams having the first wavelength λ1 to emit sideways from the optical fiber and to super-impose at a first particular focal point or focal region that is located at a first distance D1 from an outer layer of the optical fiber, wherein the first set of laser beams having the first wavelength λ1 provides a first level of energy E1 at said first particular focal point or focal region that is located at said first distance D1; and also, (II) a second, different, set of laser beams having a second, different, wavelength λ2, having a second, different, absorption coefficient A2 indicating the level of absorption by human body tissue, wherein the second set of laser beams having the second wavelength λ2 are diverted within the optical fiber by said same particular optical element, which causes the second set of laser beams having the second wavelength λ2 to emit sideways from the optical fiber and to super-impose at a second, different, particular focal point or focal region that is located at a second, different, distance D2 from the outer layer of the optical fiber, wherein the second set of laser beams having the second wavelength λ2 provides a second, different, level of energy E2 at said second particular focal point or focal region that is located at said second distance D2.

In some embodiments, the medical device is configured to perform an in vivo laser-based medical procedure by emitting laser energy sideways relative to a general direction of propagation of laser energy within the optical fiber.

In some embodiments, an entirety of the laser beams that enter into the optical fiber via its proximal end, propagate within the optical fiber and are then emitted sideways via a side-panel or the optical fiber; and none of the laser beams that enter into the optical fiber via its proximal end, exit the optical fiber via a cap or tip located at the distal end of the optical fiber.

Some embodiments include a medical device, comprising: an optical fiber configured to be inserted, at least partially, into a body of a human. The optical fiber is flexible, and has a proximal end that remains outside the body of the human, and a distal end that is controllably movable within the human body by one or more operations of a medical device operator that are selected from the group consisting of: pushing, pulling, bending, spinning, turning, flexing. The optical fiber is configured to receive, at said proximal end, one or more laser beams that are generated by a laser beam generator that is operably associated with said optical fiber. At least one region of the optical fiber includes, at an internal side of said optical fiber, at least one optical element that causes at least a portion of said one or more laser beams to exit the optical fiber via a side-wall of the optical sideways, and causes directional emission of laser energy sideways or perpendicularly to a longest dimension of said optical fiber, instead of through a cap or tip located at a distal end of said optical fiber. The medical device is configured to perform an in vivo laser-based medical procedure by emitting laser energy sideways relative to a general direction of propagation of laser energy within the optical fiber. The medical device comprises an encapsulation sleeve, that encapsulates at least a segment of the optical fiber; wherein said segment of the optical fiber is intended to be inserted into the body of said human. The encapsulation sleeve comprises, at said segment, an inflatable balloon chamber, that is controllably inflatable in vivo by receiving an inflow of a fluid that causes inflation of said inflatable balloon chamber and thus causes a modification of a distance between (i) an inner wall of the optical fiber and (ii) an in vivo point-of-interest to be medically treated, and in turn causes a modification of a power density of laser-based energy that reaches said in vivo point-of-interest. Said fluid comprises one or more of: a gas, a liquid, a solution, a saline solution, a solution of water and sugar, a solution of water and salt.

Some embodiments include a medical device, comprising: an optical fiber configured to be inserted, at least partially, into a body of a human. The optical fiber is flexible, and has a proximal end that remains outside the body of the human, and a distal end that is controllably movable within the human body by one or more operations of a medical device operator that are selected from the group consisting of: pushing, pulling, bending, spinning, turning, flexing. The optical fiber is configured to receive, at said proximal end, one or more laser beams that are generated by a laser beam generator that is operably associated with said optical fiber. At least one region of the optical fiber includes, at an internal side of said optical fiber, at least one optical element that causes at least a portion of said one or more laser beams to exit the optical fiber via a side-wall of the optical sideways, and causes directional emission of laser energy sideways or perpendicularly to a longest dimension of said optical fiber, instead of through a cap or tip located at a distal end of said optical fiber. The medical device is configured to perform an in vivo laser-based medical procedure by emitting laser energy sideways relative to a general direction of propagation of laser energy within the optical fiber. The medical device comprises an encapsulation sleeve, that encapsulates at least a segment of the optical fiber; wherein said segment of the optical fiber is intended to be inserted into the body of said human. The encapsulation sleeve comprises a non-inflatable fluid-holding canal, that is configured to receive an inflow of a fluid that causes a modification of a focal point at which two or more laser beams intersect and super-impose after they exit sideways via a wall of said optic fiber and via said fluid-holding canal; wherein said fluid comprises one or more of: a gas, a liquid, a solution, a saline solution, a solution of water and sugar, a solution of water and salt.

Some embodiments include a method of operating a medical device, the method comprising: providing and/or manufacturing and/or producing an optical fiber that is configured to be inserted, at least partially, into a body of a human; wherein the optical fiber is flexible, and has a proximal end that remains outside the body of the human, and a distal end that is controllably movable within the human body by one or more operations of a medical device operator that are selected from the group consisting of: pushing, pulling, bending, spinning, turning, flexing; providing into the proximal end of said optical fiber, one or more laser beams that are generated by a laser beam generator that is operably associated with said optical fiber; via at least one optical element, that is located at an internal side of said optical fiber, performing deflecting and/or refracting of one or more laser beams that propagate through said optical fiber in accordance with a particular laser-energy distribution and emission scheme, and directing at least one laser beam to exit said optical fiber sideways relative to a long axis of said optical fiber through a side-wall of the optical fiber and to provide laser energy to an in-vivo location that is located sideways relative to said optical fiber.

The terms “plurality” and “a plurality”, as used herein, include, for example, “multiple” or “two or more”. For example, “a plurality of items” includes two or more items.

References to “one embodiment”, “an embodiment”, “demonstrative embodiment”, “various embodiments”, “some embodiments”, and/or similar terms, may indicate that the embodiment(s) so described may optionally include a particular feature, structure, or characteristic, but not every embodiment necessarily includes the particular feature, structure, or characteristic. Furthermore, repeated use of the phrase “in one embodiment” does not necessarily refer to the same embodiment, although it may. Similarly, repeated use of the phrase “in some embodiments” does not necessarily refer to the same set or group of embodiments, although it may.

As used herein, and unless otherwise specified, the utilization of ordinal adjectives such as “first”, “second”, “third”, “fourth”, and so forth, to describe an item or an object, merely indicates that different instances of such like items or objects are being referred to; and does not intend to imply as if the items or objects so described must be in a particular given sequence, either temporally, spatially, in ranking, or in any other ordering manner.

Functions, operations, components and/or features described herein with reference to one or more embodiments, may be combined with, or may be utilized in combination with, one or more other functions, operations, components and/or features described herein with reference to one or more other embodiments. Some embodiments may comprise any possible combinations, re-arrangements, assembly, re-assembly, or other utilization of some or all of the modules or functions or components that are described herein, even if they are discussed in different locations or different chapters of the above discussion, or even if they are shown across different drawings or multiple drawings.

While certain features of some demonstrative embodiments have been illustrated and described herein, various modifications, substitutions, changes, and equivalents may occur to those skilled in the art. Accordingly, the claims are intended to cover all such modifications, substitutions, changes, and equivalents.

1. A medical device, comprising:
an optical fiber configured to be inserted, at least partially, into a body of a human;
wherein the optical fiber is flexible, and has a proximal end that remains outside the body of the human, and a distal end that is controllably movable within the human body by one or more operations of a medical device operator that are selected from the group consisting of: pushing, pulling, bending, spinning, turning, flexing;
wherein the optical fiber is configured to receive, at said proximal end, one or more laser beams that are generated by a laser beam generator that is operably associated with said optical fiber;
wherein at least one region of the optical fiber includes, at an internal side of said optical fiber, at least one optical element selected from the group consisting of: (i) a refracting optical element that refracts one or more laser beams that propagate within the optical fiber, (ii) a deflecting optical element that deflects one or more laser beams that propagate within the optical fiber, (iii) an optical element that deflects one or more laser beams that propagate within the optical fiber and refracts one or more other laser beams that propagate within the optical fiber;
wherein the at least one optical element, that is located at said internal side of said optical fiber, performs deflecting and/or refracting of one or more laser beams that propagate through said optical fiber in accordance with a particular laser-energy distribution and emission scheme, and directs at least one laser beam to exit said optical fiber sideways relative to a long axis of said optical fiber through a side-wall of the optical fiber and to provide laser energy to an in-vivo location that is located sideways relative to said optical fiber.

2. The medical device of claim 1,
wherein the at least one optical element, that is located at said internal side of said optical fiber, is configured to direct two or more laser beams to exit sideways relative to the long axis of said optical fiber, and to intersect and super-impose at a particular distance sideways relative to the long axis of said optical fiber.

3. The medical device of claim 1,
wherein the at least one optical element, that is located at said internal side of said optical fiber, is configured to direct one or more laser beams to exit sideways from said optical fiber towards a first direction, and is configured to direct one or more other laser beams to exit sideways from said optical fiber towards a second, different, direction.

4. The medical device of claim 1,
wherein the at least one optical element comprises:
(I) a first optical element,
that is located at a first location of the internal side of the optical fiber,
and is configured to direct one or more laser beams to exit sideways from said optical fiber towards a first direction;
and
(II) a second optical element,
that is located at a second, different, location of the internal side of the optical fiber,
and is configured to direct one or more laser beams to exit sideways from said optical fiber towards a second, different, direction.

5. The medical device of claim 1,
wherein the at least one optical element comprises:
(I) a first optical element,
that is located at a first location of the internal side of the optical fiber,
and is configured to direct two or more laser beams to exit sideways from said optical fiber towards a first direction, and to intersect and super-impose at a first intersection region that is located at a first distance from said optical fiber;
and
(II) a second optical element,
that is located at a second, different, location of the internal side of the optical fiber,
and is configured to direct two or more laser beams to exit sideways from said optical fiber towards a second, different, direction, and to intersect and super-impose at a second, different, intersection region that is located at a second, different, distance from said optical fiber.

6. The medical device of claim 1,
wherein the at least one optical element comprises:
(I) a first optical element, that is located at a first location of the internal side of the optical fiber,
and is configured
to direct two or more laser beams to exit sideways from said optical fiber towards a first direction,
and to intersect and super-impose at a first intersection region that is located at a first distance from said optical fiber;
and to output sideways towards said first direction N percent of an entirety of laser energy that entered the optical fiber via the proximal end;
and
(II) a second optical element,
that is located at a second, different, location of the internal side of the optical fiber,
and is configured to direct two or more laser beams to exit sideways from said optical fiber towards a second, different, direction,
and to intersect and super-impose at a second, different, intersection region that is located at a second, different, distance from said optical fiber,
and to output sideways towards said second direction M percent of an entirety of laser energy that entered the optical fiber via the proximal end;
wherein N is smaller than 100, wherein M is smaller than 100,
wherein N is different than M.

7. The medical device of claim 1,
wherein the distal end of the optical fiber comprises a cap element or a tip element;
wherein a laser energy E enters said optical fiber via the proximal end;
wherein the at least one optical element, located at the internal side of the optical fiber, is configured to divert N1 percent of said laser energy to exit sideways relative to said optical fiber;
wherein N2 percent of said laser energy is routed by said optical fiber and exits said optical fiber at the distal end, via said cap element or tip element, in a forward direction and not in a sideway direction relative to the optical fiber;
wherein N1 is smaller than 100; wherein N2 is smaller than 100;
wherein N1 is different than N2.

8. The medical device of claim 1,
wherein the distal end of the optical fiber comprises a cap element or a tip element;
wherein a laser energy E enters said optical fiber via the proximal end;
wherein the at least one optical element comprises a first optical element and a second optical element, that are located at two different locations within the optical fiber;
wherein the first optical element within the optical fiber, is configured to divert N1 percent of said laser energy to exit sideways relative to said optical fiber, towards a first sideways direction;
wherein the second optical element within the optical fiber, is configured to divert N2 percent of said laser energy to exit sideways relative to said optical fiber, towards a second, different, sideways direction;
wherein N3 percent of said laser energy is routed by said optical fiber and exits said optical fiber at the distal end, via said cap element or tip element, in a forward direction and not in a sideway direction relative to the optical fiber;
wherein N1 is smaller than 100; wherein N2 is smaller than 100;
wherein N3 is smaller than 100;
wherein N1 is different than N2.

9. The medical device of claim 1,
wherein the at least one optical element comprises at least:
an inwardly-facing wedge or an inwardly-facing protrusion, that diverts at least some of laser beams that enter the optical fiber, to exit the optical fiber sideways and not through the distal end of the optical fiber.

10. The medical device of claim 1,
wherein the at least one optical element comprises at least:
(I) a first inwardly-facing slanted wedge, that diverts a first portion of laser energy that enters the optical fiber, to exit the optical fiber sideways towards a first sideways direction relative to the optical fiber, and not through the distal end of the optical fiber;
(II) a second inwardly-facing slanted wedge, that diverts a second, different, portion of laser energy that enters the optical fiber, to exit the optical fiber sideways towards a second, different, sideways direction relative to the optical fiber, and not through the distal end of the optical fiber.

11. The medical device of claim 1,
wherein the at least one optical element comprises at least:
an inwardly-facing, generally spiral or generally helical, elongated protrusion,
that spirals internally within the optical fiber as an internal spiral protrusion or as an internal helical protrusion, and that provides at least one of: (i) a continuum of modification of optical properties of laser energy that propagates within the optical fiber, (ii) a continuum of guidance of laser beams that propagate internally within the optical fiber;
and that diverts at least some of laser beams that enter the optical fiber, to exit the optical fiber sideways and not through the distal end of the optical fiber.

12. The medical device of claim 1,
wherein the at least one optical element comprises two or more optical elements,
that are located internally within the optical fiber,
and that divert laser energy to exit non-symmetrically and sideways from said optical fiber;
wherein a first portion of laser energy that enters the optical fiber via the proximal end, exits sideways from said optical fiber, not via the distal end of the optical fiber, and towards a first sideways direction;
wherein a second, different, portion of laser energy that enters the optical fiber via the proximal end, exits sideways from said optical fiber, not via the distal end of the optical fiber, and towards a second, different, sideways direction that is non-symmetrical relative to said first sideways direction.

13. The medical device of claim 1,
wherein the at least one optical element is located internally within the optical fiber, and diverts a first portion of laser energy that entered the optical fiber via the proximal end, to exit the optical fiber sideways and to enable medical treatment via laser energy via a side-wall of the optical fiber and not via the distal end of the optical fiber;
wherein the at least one optical element also diverts a second portion of laser energy, that entered the optical fiber via the proximal end, to exit the optical fiber in a forward direction via the distal end of the optical fiber.

14. The medical device of claim 1, further comprising:
an encapsulation sleeve, that encapsulates at least a segment of the optical fiber,
wherein said segment of the optical fiber is intended to be inserted into the body of said human;
wherein the encapsulation sleeve comprises a non-inflatable fluid-holding canal,
that is configured to receive an inflow of a fluid that causes a modification of a focal point at which two or more laser beams intersect and super-impose after they exit sideways via a wall of said optic fiber and via said fluid-holding canal;
wherein said fluid comprises one or more of: a gas, a liquid, a solution, a saline solution, a solution of water and sugar, a solution of water and salt.

15. The medical device of claim 14, further comprising:
a control unit that is connected ex vivo to the optical fiber,
and that selectively or temporarily pumps a particular fluid into said non-inflatable fluid-holding canal or out of said non-inflatable fluid-holding canal,
and selective or temporarily causes a modification of the focal point at which two or more laser beams intersect and super-impose after they exit sideways via the wall of said optic fiber and via said non-inflatable fluid-holding canal.

16. The medical device of claim 14, further comprising:
a control unit that is connected ex vivo to the optical fiber,
and that selectively or temporarily pumps a particular material into said non-inflatable fluid-holding canal or out of said non-inflatable fluid-holding canal, and thus modifies a concentration of a solution that is held within said non-inflatable fluid-holding canal,
and selective or temporarily causes a modification of the focal point at which two or more laser beams intersect and super-impose after they exit sideways via the wall of said optic fiber and via said non-inflatable fluid-holding canal.

17. The medical device of claim 1, further comprising:
an encapsulation sleeve, that encapsulates at least a segment of the optical fiber,
wherein said segment of the optical fiber is intended to be inserted into the body of said human;
wherein the encapsulation sleeve comprises, at said segment, an inflatable balloon chamber,
that is controllably inflatable in vivo by receiving an inflow of a fluid that causes inflation of said inflatable balloon chamber and thus causes a modification of a distance between (i) an inner wall of the optical fiber and (ii) an in vivo point-of-interest to be medically treated, and in turn causes a modification of a power density of laser-based energy that reaches said in vivo point-of-interest;
wherein said fluid comprises one or more of: a gas, a liquid, a solution, a saline solution, a solution of water and sugar, a solution of water and salt.

18. The medical device of claim 1,
wherein the at least one optical element comprises at least:
(a) a first curved optical element, that (a1) diverts a first portion of laser energy that enters the optical fiber, to exit the optical fiber sideways towards a first sideways direction relative to the optical fiber, and not through the distal end of the optical fiber; and that (a2) allows a second portion of the laser energy that enters the optical fiber, to proceed with non-modified propagation towards a second, different, curved optical element that is located further along said optical fiber;
(b) said second, different, curved optical element, that is located further along said optical fiber, and that receives said second portion of the laser energy, and that diverts said second portion of laser energy to exit the optical fiber sideways towards a second, different, sideways direction relative to the optical fiber, and not through the distal end of the optical fiber;
wherein the first portion of laser energy that exited sideways from the optical fiber due to the first curved optical element, and the second portion of laser energy that exited sideways from the optical fiber due to the second curved optical element, collide and super-impose at an in vivo region-of-interest that is a focal region and is not a singular focal point.

19. The medical device of claim 1,
wherein the optical fiber has a non-circular or non-symmetrical cross-section, (i) which improves mechanical control of bending operations performed by an operator of the medical device, (ii) and which enables the operator of the medical device efficient understanding of the in-vivo spatial orientation of the optical fiber.

20. The medical device of claim 19,
wherein the optical fiber has an oval or egg-shaped cross-section.

21. The medical device of claim 19,
wherein the optical fiber has a cross section that consists of: a straight line, and an arc of less than 300 degrees.

22. The medical device of claim 1,
wherein the optical fiber is configured, due to incorporation of said at least one optical element therein, to emit directional, sideways, non-forward directed, laser energy,
which is emitted sideways through a side-wall of the optical fiber and not through a cap or tip located at the distal end of the optical fiber,
in accordance with a weighted energy emission scheme that defines: (i) that N1 percent of laser energy that entered the optical fiber via the proximal end is emitted sideways through a first location of a side-wall of the optical fiber towards a first sideways and non-forward direction, and (ii) that N2 percent of laser energy that entered the optical fiber via the proximal end is emitted sideways through a second location of the side-wall of the optical fiber towards a second sideways and non-forward direction, and (iii) that N3 percent of laser energy that entered the optical fiber via the proximal end is emitted in a forward direction relative to the optical fiber and via the cap or tip of the distal end of the optical fiber;
wherein N1 is different from N2 and is different from N3;
wherein N2 is different from N1 and is different from N3;
wherein N3 is different from N1 and is different from N2.

23. The medical device of claim 1,
wherein the optical fiber is configured, due to incorporation of said at least one optical element therein,
to emit directional, sideways, non-forward directed, laser energy,
that performs a medical procedure at a body-location that is located sideways relative to a longest dimension of the optical fiber, and that is not located at a forward direction relative to the longest dimension of the optical fiber; and that provides laser energy to said body-location that is located sideways relative to the longest dimension of the optical fiber in accordance with a pre-defined or dynamically-modifiable energy distribution scheme.

24. The medical device of claim 1,
wherein the optical fiber is configured to receive, at the proximal end of the optical fiber, from said laser beam generator that is operably associated with said optical fiber:
(I) a first set of laser beams having a first wavelength λ1,
having a first absorption coefficient A1 indicating a level of absorption by human body tissue,
wherein the first set of laser beams having the first wavelength λ1 are diverted within the optical fiber by a first set of optical elements, which cause the first set of laser beams having the first wavelength λ1 to emit sideways from the optical fiber and to super-impose at a first particular focal point or focal region that is located at a first distance D1 from an outer layer of the optical fiber,
wherein the first set of laser beams having the first wavelength λ1 provides a first level of energy E1 at said first particular focal point or focal region that is located at said first distance D1;
(II) a second, different, set of laser beams having a second, different, wavelength λ2,
having a second, different, absorption coefficient A2 indicating the level of absorption by human body tissue,
wherein the second set of laser beams having the second wavelength λ2 are diverted within the optical fiber by a second set of optical elements, which cause the second set of laser beams having the second wavelength λ2 to emit sideways from the optical fiber and to super-impose at a second, different, particular focal point or focal region that is located at a second, different, distance D2 from the outer layer of the optical fiber,
wherein the second set of laser beams having the second wavelength λ2 provides a second, different, level of energy E2 at said second particular focal point or focal region that is located at said second distance D2.

25. The medical device of claim 1,
wherein the optical fiber is configured to receive, at the proximal end of the optical fiber, from said laser beam generator that is operably associated with said optical fiber:
(I) a first set of laser beams having a first wavelength λ1,
having a first absorption coefficient A1 indicating a level of absorption by human body tissue,
wherein the first set of laser beams having the first wavelength λ1 are diverted within the optical fiber by a particular optical element, which causes the first set of laser beams having the first wavelength λ1 to emit sideways from the optical fiber and to super-impose at a first particular focal point or focal region that is located at a first distance D1 from an outer layer of the optical fiber,
wherein the first set of laser beams having the first wavelength λ1 provides a first level of energy E1 at said first particular focal point or focal region that is located at said first distance D1:
(II) a second, different, set of laser beams having a second, different, wavelength λ2,
having a second, different, absorption coefficient A2 indicating the level of absorption by human body tissue,
wherein the second set of laser beams having the second wavelength λ2 are diverted within the optical fiber by said same particular optical element, which causes the second set of laser beams having the second wavelength λ2 to emit sideways from the optical fiber and to super-impose at a second, different, particular focal point or focal region that is located at a second, different, distance D2 from the outer layer of the optical fiber,
wherein the second set of laser beams having the second wavelength λ2 provides a second, different, level of energy E2 at said second particular focal point or focal region that is located at said second distance D2.

26. The medical device of claim 1,
wherein the medical device is configured to perform an in vivo laser-based medical procedure by emitting laser energy sideways relative to a general direction of propagation of laser energy within the optical fiber.

27. The medical device of claim 1,
wherein an entirety of the laser beams that enter into the optical fiber via its proximal end, propagate within the optical fiber and are then emitted sideways via a side-panel or the optical fiber;
and wherein none of the laser beams that enter into the optical fiber via its proximal end, exit the optical fiber via a cap or tip located at the distal end of the optical fiber.

28. A medical device, comprising:
an optical fiber configured to be inserted, at least partially, into a body of a human;
wherein the optical fiber is flexible, and has a proximal end that remains outside the body of the human, and a distal end that is controllably movable within the human body by one or more operations of a medical device operator that are selected from the group consisting of:
pushing, pulling, bending, spinning, turning, flexing;
wherein the optical fiber is configured to receive, at said proximal end, one or more laser beams that are generated by a laser beam generator that is operably associated with said optical fiber;
wherein at least one region of the optical fiber includes, at an internal side of said optical fiber, at least one optical element that causes at least a portion of said one or more laser beams to exit the optical fiber via a side-wall of the optical sideways, and causes directional emission of laser energy sideways or perpendicularly to a longest dimension of said optical fiber, instead of through a cap or tip located at a distal end of said optical fiber;
wherein the medical device is configured to perform an in vivo laser-based medical procedure by emitting laser energy sideways relative to a general direction of propagation of laser energy within the optical fiber;
wherein the medical device comprises an encapsulation sleeve, that encapsulates at least a segment of the optical fiber,
wherein said segment of the optical fiber is intended to be inserted into the body of said human;
wherein the encapsulation sleeve comprises, at said segment, an inflatable balloon chamber,
that is controllably inflatable in vivo by receiving an inflow of a fluid that causes inflation of said inflatable balloon chamber and thus causes a modification of a distance between (i) an inner wall of the optical fiber and (ii) an in vivo point-of-interest to be medically treated, and in turn causes a modification of a power density of laser-based energy that reaches said in vivo point-of-interest;
wherein said fluid comprises one or more of: a gas, a liquid, a solution, a saline solution, a solution of water and sugar, a solution of water and salt.

29. A medical device, comprising:
an optical fiber configured to be inserted, at least partially, into a body of a human;
wherein the optical fiber is flexible, and has a proximal end that remains outside the body of the human, and a distal end that is controllably movable within the human body by one or more operations of a medical device operator that are selected from the group consisting of: pushing, pulling, bending, spinning, turning, flexing;
wherein the optical fiber is configured to receive, at said proximal end, one or more laser beams that are generated by a laser beam generator that is operably associated with said optical fiber;
wherein at least one region of the optical fiber includes, at an internal side of said optical fiber, at least one optical element that causes at least a portion of said one or more laser beams to exit the optical fiber via a side-wall of the optical sideways, and causes directional emission of laser energy sideways or perpendicularly to a longest dimension of said optical fiber, instead of through a cap or tip located at a distal end of said optical fiber;
wherein the medical device is configured to perform an in vivo laser-based medical procedure by emitting laser energy sideways relative to a general direction of propagation of laser energy within the optical fiber;
wherein the medical device comprises an encapsulation sleeve, that encapsulates at least a segment of the optical fiber,
wherein said segment of the optical fiber is intended to be inserted into the body of said human;
wherein the encapsulation sleeve comprises a non-inflatable fluid-holding canal,
that is configured to receive an inflow of a fluid that causes a modification of a focal point at which two or more laser beams intersect and super-impose after they exit sideways via a wall of said optic fiber and via said fluid-holding canal;
wherein said fluid comprises one or more of: a gas, a liquid, a solution, a saline solution, a solution of water and sugar, a solution of water and salt.

30. A method of operating a medical device, the method comprising:
providing an optical fiber that is configured to be inserted, at least partially, into a body of a human;
wherein the optical fiber is flexible, and has a proximal end that remains outside the body of the human, and a distal end that is controllably movable within the human body by one or more operations of a medical device operator that are selected from the group consisting of: pushing, pulling, bending, spinning, turning, flexing;
providing into the proximal end of said optical fiber, one or more laser beams that are generated by a laser beam generator that is operably associated with said optical fiber;
via at least one optical element, that is located at an internal side of said optical fiber, performing deflecting and/or refracting of one or more laser beams that propagate through said optical fiber in accordance with a particular laser-energy distribution and emission scheme, and directing at least one laser beam to exit said optical fiber sideways relative to a long axis of said optical fiber through a side-wall of the optical fiber and to provide laser energy to an in-vivo location that is located sideways relative to said optical fiber.
